SEQUENCE LISTING (2 sequences)

SEQ ID NO: 1 - DNA, 18 bp, Artificial Sequence
  Synthetic polynucleotide
tagctgcacgctgccgtc

SEQ ID NO: 2 - DNA, 18 bp, Artificial Sequence
  Synthetic polynucleotide
tagctgcacgctgccgtc

(19) United States
(12) Patent Application Publication
(10) Pub. No.: US 2012/0244230 A1
(43) Pub. Date: Sep. 27, 2012

(54) POLYVALENT POLYNUCLEOTIDE NANOPARTICLE CONJUGATES AS DELIVERY VEHICLES FOR A CHEMOTHERAPEUTIC AGENT

(75) Inventors: Chad A. Mirkin, Wilmette, IL (US); David A. Giljohann, Chicago, IL (US); Weston L. Daniel, Evanston, IL (US); Stephen J. Lippard, Cambridge, MA (US); Shanta Dhar, Athens, GA (US)

(73) Assignees: MASSACHUSETTS INSTITUTE OF TECHNOLOGY, Cambridge, MA (US); NORTHWESTERN UNIVERSITY, Evanston, IL (US)

(21) Appl. No.: 13/393,454

(22) PCT Filed: Sep. 1, 2010
(86) PCT No.: PCT/US10/47591

§ 371 Date: Jun. 13, 2012

Related U.S. Application Data
(60) Provisional application No. 61/238,930, filed on Sep. 1, 2009.

(51) Int. Cl. C07H 21/00; C12N 5/02; A61P 35/00; C07H 21/04; C07H 21/02; A61K 33/24; C07K 2/00; B82Y 5/00

(52) U.S. Cl. 424/649; 435/375; 530/322; 536/23.1; 536/24.3; 977/773; 977/774; 977/810; 977/906

(57) ABSTRACT

The present invention is directed to compositions and methods of delivering a chemotherapeutic agent via a polynueleotide-functionalized nanoparticle (PN-NP).

### CROSS-REFERENCE TO RELATED APPLICATIONS

This application claims the priority benefit under 35 U.S.C. §119(e) of U.S. Provisional Application No. 61/238,930, filed Sep. 1, 2009, the disclosure of which is incorporated herein by reference in its entirety.

### STATEMENT OF GOVERNMENT INTEREST

This invention was made with government support under Grant Number 5U54 CA119341, awarded by the NIH (NCI), and Grant Number CA034992, awarded by the NIH (NCI). The government has certain rights in the invention.

### FIELD OF THE INVENTION

The present invention is directed to compositions and methods of delivering a chemotherapeutic agent via a polynucleotide-functionalized nanoparticle (PN-NP).

### BACKGROUND OF THE INVENTION

Synthetic delivery systems have great potential for overcoming problems associated with systemic toxicity that accompanies chemotherapy, including but not limited to platinum-based treatment [Haxton et al., J. Pharm. Sci. 98: 2299-2316 (2009)]. Finding successful candidates and strategies for the delivery of platinum anticancer drugs has been a subject of extensive research. Delivery systems provide a variety of functions, such as improving poor solubility, enhancing in vivo stability, and optimizing biodistribution and pharmacokinetics [Ould-Ouali, et al., J. Controlled Release 102: 657-668 (2005)].

Chemotherapy is often based on the use of drugs that are selectively toxic (cytotoxic) to cancer cells. Several general classes of chemotherapeutic drugs have been developed. A first class, antimetabolite drugs, includes drugs that interfere with nucleic acid synthesis, protein synthesis, and other vital metabolic processes. Another class, genotoxic drugs, inflicts damage on cellular nucleic acids, including DNA. Two widely used genotoxic anticancer drugs that have been shown to damage cellular DNA by producing crosslinks therein are cisplatin [cis-diamminedichloroplatinum(II)] and carboplatin [diammine(1,1-cyclobutanedicarboxylato)-platinum(II)]. Cisplatin and carboplatin currently are used in the treatment of selected, diverse neoplasms of epithelial and mesenchymal origin, including carcinomas and sarcomas of the respiratory, gastrointestinal and reproductive tracts, of the central nervous system, and of squamous origin in the head and neck. Cisplatin currently is preferred for the management of testicular carcinoma and in many instances produces a lasting remission.

Whereas cisplatin [Jamieson et al., J. Chem. Rev. 99: 2467-2498 (1999); Rosenberg et al., Nature 222: 385-6 (1969)] is one of the most effective anticancer drugs, its side effects include kidney toxicity, nausea, hearing impairment, and irreversible peripheral nerve damage [Hartmann et al., Int. J. Cancer 83: 866-9 1999); Laurell et al., Laryngoscope 100: 724-34 (1990); Thompson et al., Cancer 54: 1269-75 (1984)]. To reduce such side effects and target tumor tissue, researchers [Dhar et al., Proc. Natl. Acad. Sci. U.S.A. 105: 17356-17361 (2008); Dhar et al., J. Am. Chem. Soc. 130: 11467-11476 2008); Feazell et al., J. Am. Chem. Soc. 129: 8438-8439 (2007); Rieter et al., J. Am. Chem. Soc. 130: 11584-11585 (2008); Sood et al., Bioconjugate Chem. 17: 1270-1279 (2006)] have been investigating a variety of nanoparticulate delivery vehicles over the past several years. Pt(IV) complexes provide an attractive alternative to Pt(II) compounds because their inertness results in fewer side effects. Pt(II)-based anticancer drugs are associated with higher reactivity and thus lower biological stability. Pt(IV) complexes are reduced in the intracellular milieu to yield the cytotoxic Pt(II) species through reductive elimination of axial ligands [Ciandomenico et al., Inorg. Chem. 34: 1015-21 (1995)]. Thus, Pt(IV) complexes provide an attractive alternative to the existing portfolio of Pt(II) drugs.

### SUMMARY OF THE INVENTION

The present disclosure describes compositions and methods that combine the properties of polynucleotide-functionalized nanoparticles (PN-NPs) and a chemotherapeutic agent into a single agent for drug delivery. Thus, in some aspects the present disclosure provides a composition comprising a PN-NP and a platinum coordination complex, wherein the platinum coordination complex is attached to the polynucleotide, and wherein the platinum coordination complex is activated upon cell uptake. In some aspects, the platinum coordination complex is platinum(IV) (Pt(IV)) or is platinum(II) (Pt(II)).

Compositions provided by the disclosure include, in various aspects, those wherein the nanoparticle is metallic. In further aspects, compositions are provided wherein the nanoparticle is a colloidal metal. In various aspects of the disclosure, the nanoparticle is selected from the group consisting of a gold nanoparticle, a silver nanoparticle, a platinum nanoparticle, an aluminum nanoparticle, a palladium nanoparticle, a copper nanoparticle, a cobalt nanoparticle, an indium nanoparticle, and a nickel nanoparticle. In one specific aspect, the nanoparticle is a gold nanoparticle (AuNP).

In various embodiments, the activation of the platinum coordination complex results in an increase in cytotoxicity. In one aspect, the increase in cytotoxicity is about 2-fold relative to a platinum coordination complex that is not attached to a polynucleotide, wherein the polynucleotide is functionalized on a nanoparticle, and wherein the increase in cytotoxicity is measured using an in vitro cell culture assay. In various aspects, the in vitro cell culture assay is a (3-(4,5-dimethylthiazol-2-yl)-2,5-diphenyltetrazolium bromide) (MIT) assay.

In some embodiments, it is contemplated that more than one platinum coordination complex is attached to the polynucleotide. In various aspects, the polynucleotide is DNA, RNA, or a modified polynucleotide and in various embodiments, the polynucleotide comprises about 5 nucleotides to about 100 nucleotides, or the polynucleotide comprises about 10 nucleotides to about 50 nucleotides. In a specific embodiment the polynucleotide comprises about 18 nucleotides. Polynucleotides contemplated are double-stranded or single-stranded.

In a further embodiment, the composition comprising a polynucleotide-functionalized nanoparticle further comprises a second polynucleotide. In some aspects, the second polynucleotide is attached to the nanoparticle, and in further aspects the second polynucleotide further comprises a detectable marker. In aspects where the second polynucleotide further comprises a detectable marker, it is contemplated that in further aspects the detectable marker is selected from the group consisting of a fluorophore, an isotope, a contrast agent, a redox active probe, a nanoparticle, a polypeptide, a peptide, a small molecule, a metal, a metabolic group and a quantum dot.

In further embodiments, the second polynucleotide is sufficiently complementary to a target polynucleotide to hybridize to the target polynucleotide. In some aspects, the target polynucleotide is DNA or RNA, and in further aspects the target polynucleotide is in a target cell. It is further contemplated that, in various aspects, the polynucleotide and the second polynucleotide are each sufficiently complementary to hybridize to a different target polynucleotide in the target cell.

An object of the present disclosure is the delivery of a composition comprising a nanoparticle to a target cell. Accordingly, it is contemplated that in various aspects the composition further comprises a targeting moiety. The targeting moiety is any molecular structure that allows or assists the composition to be preferentially delivered to a target cell as defined herein relative to a cell that is not targeted. In various aspects, the targeting moiety can be attached to the nanoparticle or to a polynucleotide that is functionalized on the nanoparticle. In further aspects, the targeting moiety is associated with the nanoparticle, and in still further aspects the targeting moiety is co-administered with a composition of the disclosure.

Thus, the disclosure provides a method comprising delivering a composition as described herein to a target cell. According to methods of the disclosure, delivery of a composition as described herein results, in some embodiments, in activation of the chemotherapeutic agent. In some aspects, activation results in an increase cytotoxicity.

According to the present disclosure, the target cell, in one aspect, is a cancer cell. In various embodiments, the cancer is selected from the group consisting of liver, pancreatic, stomach, colorectal, prostate, testicular, renal cell, breast, bladder, ureteral, brain, lung, connective tissue, hematological, cardiovascular, lymphatic, skin, bone, eye, nasopharyngeal, laryngeal, esophagus, oral membrane, tongue, thyroid, parotid, mediastinum, ovary, uterus, adnexal, endometrial, cervical, small bowel, appendix, carcinoid, gall bladder, pituitary, cancer arising from metastatic spread, and cancer arising from endodermal, mesodermal or ectodermally-derived tissues.

Further aspects of the invention will become apparent from the detailed description provided below. However, it should be understood that the following detailed description and examples, while indicating various embodiments of the invention, are given by way of illustration only since various changes and modifications within the spirit and scope of the invention will become apparent to those skilled in the art from this detailed description.

### DESCRIPTION OF THE DRAWINGS

FIG. 1 depicts UV Vis spectra of DNA-Au NP and Pt-DNA-Au NP.

FIG. 2 depicts cyclic voltammograms of 1 in phosphate buffer-0.1M KCl of pH 7.4 with varied scan rates (top). Plot of reduction peak potential maxima of 1 at pH 7.4 as a function of scan rate (bottom).

FIG. 3 depicts cyclic voltammograms of 1 in phosphate buffer-0.1 M KCl of pH 6.0 with varied scan rates (top). Plot of reduction peak potential maxima of 1 at pH 6.0 as a function of scan rate (bottom).

FIG. 4 depicts cytotoxicity profiles of Pt-DNA-Au NP (•), cisplatin (□), 1 (Δ) with U2OS, A549, HeLa, and PC3 cells.

### DETAILED DESCRIPTION OF THE INVENTION

As described above, the disclosure provides compositions and methods for delivering a polynucleotide-functionalized nanoparticle and a chemotherapeutic agent. The chemotherapeutic agent is attached to the polynucleotide, and it is further contemplated that the chemotherapeutic agent is, for example and without limitation, a platinum coordination complex. In some aspects, the platinum coordination complex is a platinum(IV) (Pt(IV)) or a platinum(II) (Pt(II)) prodrug.

In part, the present disclosure is directed towards a composition comprising a PN-NP and a chemotherapeutic agent. Upon entry of the composition into a target cell, the resulting chemotherapeutic agent is intended to be therapeutically effective. As used herein, “therapeutically effective” means that any one or all of the effects often associated with the in vivo biological activity of the chemotherapeutic agent occur. A benefit provided by the disclosure, then, is that the compositions described herein exhibit reduced toxicity toward normal cells while conferring their therapeutic effects on target cells. An additional benefit provided by the disclosure is the use of a polynucleotide-functionalized nanoparticle, which confers advantages including but not limited to increased cell uptake and stability.

Based in part on the approach described above, compositions comprising a PN-NP and a chemotherapeutic agent have been prepared. Compositions provided optionally comprise a PN-NP, a chemotherapeutic agent, and a targeting moiety. It will be appreciated that a composition that exhibits target specific activity has therapeutic benefit, however embodiments of the composition provided do not require the presence of a targeting agent bound to, or in association with, a PN-NP comprising the chemotherapeutic agent. Targeted delivery techniques are well known and routinely practiced in the art and include, for example and without limitation, direct injection to a solid tumor, co-administration of one or more embolic agents which localized the active agents in the composition at a desired location, and/or synthesizing the PN-NP component such that it can take exploit “leaky” vasculature locations often associated with tumors and tumor growth.

It is noted here that, as used in this specification and the appended claims, the singular forms “a,” “an,” and “the” include plural reference unless the context clearly dictates otherwise.

It is also noted that the term “about” as used herein is understood to mean approximately.

Throughout the disclosure, the term “functionalized” is used interchangeably with the terms “attached” and “bound.”

### Nanoparticles

Compositions of the present disclosure comprise nanoparticles as described herein. Nanoparticles are provided which are functionalized to have a polynucleotide attached thereto. The size, shape and chemical composition of the nanoparticles contribute to the properties of the resulting PN-NP. These properties include for example, optical properties, optoelectronic properties, electrochemical properties, electronic properties, stability in various solutions, magnetic properties, and pore and channel size variation. Mixtures of nanoparticles having different sizes, shapes and/or chemical compositions, as well as the use of nanoparticles having uniform sizes, shapes and chemical composition, and therefore a mixture of properties are contemplated. Examples of suitable particles include, without limitation, aggregate particles, isotropic (such as spherical) particles, anisotropic particles (such as non-spherical rods, tetrahedral, and/or prisms) and core-shell particles, such as those described in U.S. Pat. No. 7,238,472 and International Publication No. WO 2003/08539, the disclosures of which are incorporated by reference in their entirety.

In one embodiment, the nanoparticle is metallic, and in various aspects, the nanoparticle is a colloidal metal. Thus, in various embodiments, nanoparticles of the invention include metal (including for example and without limitation, silver, gold, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials.

Also, as described in U.S. Patent Publication No 2003/0147966, nanoparticles of the invention include those that are available commercially, as well as those that are synthesized, e.g., produced from progressive nucleation in solution (e.g., by colloid reaction) or by various physical and chemical vapor deposition processes, such as sputter deposition. See, e.g., HaVashi, Vac. Sci. Technol. A5(4):1375-84 (1987); Hayashi, Physics Today, 44-60 (1987); MRS Bulletin, January 1990, 16-47. As further described in U.S. Patent Publication No 2003/0147966, nanoparticles contemplated are alternatively produced using HAuCl₄ and a citrate-reducing agent, using methods known in the art. See, e.g., Marinakos et al., Adv. Mater. 11:34-37 (1999); Marinakos et al., Chem. Mater. 10: 1214-19 (1998); Enustun & Turkevich, J. Am. Chem. Soc. 85: 3317 (1963).

Nanoparticles can range in size from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 nm in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, about 1 nm to about 10 nm in mean diameter. In other aspects, the size of the nanoparticles is from about 5 nm to about 150 nm (mean diameter), from about 5 to about 50 nm, from about 10 to about 30 nm, from about 10 to 150 nm, from about 10 to about 100 nm, or about 10 to about 50 nm. The size of the nanoparticles is from about 5 nm to about 150 nm (mean diameter), from about 30 to about 100 nm, from about 40 to about 80 nm. The size of the nanoparticles used in a method varies as required by their particular use or application. The variation of size is advantageously used to optimize certain physical characteristics of the nanoparticles, for example, optical properties or the amount of surface area that can be functionalized as described herein.

### Targeting Moiety

The term “targeting moiety” as used herein refers to any molecular structure which assists a compound or other molecule in binding or otherwise localizing to a particular target, a target area, entering target cell(s), or binding to a target receptor. For example and without limitation, targeting moieties may include proteins, peptides, aptamers, lipids (including cationic, neutral, and steroidal lipids, virosomes, and liposomes), antibodies, lectins, ligands, sugars, steroids, hormones, and nutrients, may serve as targeting moieties. Other examples of targeting moieties are described in Lippard et al., U.S. Pat. No. 7,138,520, and Priest, U.S. Pat. No. 5,391,723, each of which is incorporated herein by reference in its entirety.

In some embodiments, the targeting moiety is a protein. The protein portion of the composition of the present disclosure is, in some aspects, a protein capable of targeting the composition to target cell. Such a targeting protein may be a protein, polypeptide, or fragment thereof that is capable of binding to a desired target site in vivo. The targeting protein of the present disclosure may bind to a receptor, substrate, antigenic determinant, or other binding site on a target cell or other target site.

A targeting protein may be modified (for example and without limitation, to produce variants and fragments of the protein), as long as the desired biological property of binding to its target site is retained. A targeting protein may be modified by using various genetic engineering or protein engineering techniques. Typically, a protein will be modified to more efficiently bind to the target cell binding site. Such modifications are known and are routine to one of skill in the art.

Examples of targeting proteins include, but are not limited to, antibodies and antibody fragments; serum proteins; fibrinolytic enzymes; peptide hormones; and biologic response modifiers. Among the suitable biologic response modifiers which may be used are lymphokines, such as interleukin (for example and without limitation, IL-1, -2, -3, -4, -5, and -6) or interferon (for example and without limitation, alpha, beta and gamma), erythropoietin, and colony stimulating factors (for example and without limitation, G-CSF, GM-CSF, and M-CSF). Peptide hormones include melanocyte stimulating hormone, follicle stimulating hormone, luteinizing hormone, and human growth hormone. Fibrinolytic enzymes include tissue-type plasminogen activator, streptokinase and urokinase. Serum proteins include human serum albumin and the lipoproteins.

Antibodies useful as targeting proteins may be polyclonal or monoclonal. A number of monoclonal antibodies (MAbs) that bind to a specific type of cell have been developed. These include MAbs specific for tumor-associated antigens in humans. Exemplary of the many MAbs that may be used are anti-TAC, or other interleukin-2 receptor antibodies; NR-ML-05, or other antibodies that bind to the 250 kilodalton human melanoma-associated proteoglycan; NR-LU-10, a pancarcinoma antibody directed to a 37-40 kilodalton pancarcinoma glycoprotein; and OVB3, which recognizes an as yet unidentified, tumor-associated antigen. Antibodies derived through genetic engineering or protein engineering may be used as well.

The antibody employed as a targeting agent in the present disclosure may be an intact molecule, a fragment thereof, or a functional equivalent thereof. Examples of antibody fragments useful in the compositions of the present disclosure are F(ab′)₂, Fab′ Fab and Fv fragments, which may be produced by conventional methods or by genetic or protein engineering.

In some embodiments, the polynucleotide portion of the present invention may serve as an additional or auxiliary targeting moiety. The oligonucleotide portion may be selected or designed to assist in extracellular targeting, or to act as an intracellular targeting moiety. That is, the polynucleotide portion may act as a DNA probe seeking out target cells. This additional targeting capability will serve to improve specificity in delivery of the composition to target cells. The oligonucleotide may additionally or alternatively be selected or designed to target the composition within target cells, while the targeting protein targets the conjugate extracellularly.

Compositions of the disclosure comprise a polynucleotide-functionalized nanoparticle and a chemotherapeutic agent, wherein the chemotherapeutic agent is attached to the polynucleotide, and wherein the chemotherapeutic agent is activated upon cell uptake. In various embodiments, a composition of the disclosure further comprises a targeting moiety. It is contemplated that the targeting moiety can, in various embodiments, be attached to the nanoparticle or a polynucleotide. In aspects wherein the targeting moiety is a polynucleotide, it is contemplated that it is attached to the nanoparticle, or is part of a polynucleotide that is conjugated to a chemotherapeutic agent. In further aspects, the targeting moiety is associated with the nanoparticle composition, and in other aspects the targeting moiety is administered before, concurrent with, or after the administration of a composition of the disclosure.

### Polynucleotides

The terms “polynucleotide” and “nucleotide” or plural forms as used herein are interchangeable with modified forms as discussed herein and otherwise known in the art. In certain instances, the art uses the term “nucleobase” which embraces naturally-occurring nucleotides as well as modifications of nucleotides that can be polymerized. Thus, nucleotide or nucleobase means the naturally occurring nucleobases adenine (A), guanine (G), cytosine (C), thymine (T) and uracil (U) as well as non-naturally occurring nucleobases such as xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C₃-C₆)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, polynucleotides also include one or more “nucleosidic bases” or “base units” which include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Polynucleotides may also include modified nucleobases. A “modified base” is understood in the art to be one that can pair with a natural base (e.g., adenine, guanine, cytosine, uracil, and/or thymine) and/or can pair with a non-naturally occurring base. Exemplary modified bases are described in EP 1 072 679 and WO 97/12896, the disclosures of which are incorporated herein by reference. Modified nucleobases include without limitation, 5-methylcytosine (5-me-(−), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one , carbazole cytidine (2H-pyrimido[4,5-b]indol-2-one), pyridoindole cytidine pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (196)); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

Nanoparticles provided that are functionalized with a polynucleotide, or modified form thereof, generally comprise a polynucleotide from about 5 nucleotides to about 100 nucleotides in length. More specifically, nanoparticles are functionalized with polynucleotide that are about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, and all polynucleotides intermediate in length of the sizes specifically disclosed to the extent that the polynucleotide is able to achieve the desired result. Accordingly, polynucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length are contemplated.

### Attachment of a Chemotherapeutic Agent

The disclosure provides PN-NPs wherein a chemotherapeutic agent is attached to the polynucleotide. Methods of attaching a drug or a chemotherapeutic agent to a polynucleotide are known in the art, and are described in Priest, U.S. Pat. No. 5,391,723, Arnold, Jr., et al., U.S. Pat. No. 5,585,481, Reed et al., U.S. Pat. No. 5,512,667 and PCT/US2006/022325, the disclosures of which are incorporated herein by reference in their entirety). Any chemotherapeutic agent may be attached to the polynucleotide, provided that the chemotherapeutic agent is relatively inactive when attached to the polynucleotide that is further attached to the nanoparticle, but is activated upon cell uptake. By “relatively inactive” is meant that the cytotoxic capability of the chemotherapeutic agent is reduced when not attached to a PN-NP as described herein compared to the cytotoxic capability of the chemotherapeutic agent when it is attached to a PN-NP. Methods for determining cytotoxic capability are known in the art, and described herein.

It is contemplated that, in some embodiments, a polynucleotide is functionalized with more than one of the same chemotherapeutic agent. In various aspects, a polynucleotide is functionalized with 2, 3, 4, 5, 6, 7, 8, 9, 10 or more chemotherapeutic agents. In further aspects, a polynucleotide is functionalized with 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more chemotherapeutic agents. It will be understood that the number of chemotherapeutic agents associated with a nanoparticle will depend on the diameter of the nanoparticle. The larger the diameter, the higher the number of polynucleotides that can be functionalized thereto, and thus the higher the number of chemotherapeutic agents that will be associated with the nanoparticle. Accordingly, it is contemplated that a nanoparticle can comprise between 1 and 10×10⁶, or between about 10 and about 1×10⁶, or between about 1000 and about 1×10⁵, or between about 5000 and about 50,000 chemotherapeutic agents. In various aspects, the disclosure contemplates that a nanoparticle can comprise about 150, or about 200, or about 250, or about 300, or about 350, or about 400, or about 450, or about 500, or about 550, or about 600, or about 650, or about 700, or about 750, or about 800, or about 850, or about 900, or about 950, or about 1000, or about 1050, or about 1100, or about 1150, or about 1200, or about 1250, or about 1300, or about 1350, or about 1400, or about 1450, or about 1500, or about 1550, or about 1600, or about 1650, or about 1700, or about 1750, or about 1800, or about 1850, or about 1900, or about 1950, or about 2000, or about 2050, or about 2100, or about 2150, or about 2200, or about 2250, or about 2300, or about 2350, or about 2400, or about 2450, or about 2500, or about 2550, or about 2600, or about 2650, or about 2700, or about 2750, or about 2800, or about 2850, or about 2900, or about 2950, or about 3000, or about 3050, or about 3100, or about 3150, or about 3200, or about 3250, or about 3300, or about 3350, or about 3400, or about 3450, or about 3500, or about 3550, or about 3600, or about 3650, or about 3700, or about 3750, or about 3800, or about 3850, or about 3900, or about 3950, or about 4000, or about 4050, or about 4100, or about 4150, or about 4200, or about 4250, or about 4300, or about 4350, or about 4400, or about 4450, or about 4500, or about 4550, or about 4600, or about 4650, or about 4700, or about 4750, or about 4800, or about 4850, or about 4900, or about 4950, or about 5000, or about 6000, or about 7000, or about 8000, or about 9000, or about 10000, or about 20000, or about 30000, or about 40000, or about 50000, or about 60000, or about 70000, or about 80000, or about 90000, or about 100000, or about 110000, or about 120000, or about 130000, or about 140000, or about 150000, or about 160000, or about 170000, or about 180000, or about 190000, or about 200000, or about 210000, or about 220000, or about 230000, or about 240000, or about 250000, or about 260000, or about 270000, or about 280000, or about 290000, or about 300000, or about 310000, or about 320000, or about 330000, or about 340000, or about 350000, or about 360000, or about 370000, or about 380000, or about 390000, or about 400000, or about 410000, or about 420000, or about 430000, or about 440000, or about 450000, or about 460000, or about 470000, or about 480000, or about 490000, or about 500000, or about 510000, or about 520000, or about 530000, or about 540000, or about 550000, or about 560000, or about 570000, or about 580000, or about 590000, or about 600000, or about 610000, or about 620000, or about 630000, or about 640000, or about 650000, or about 660000, or about 670000, or about 680000, or about 690000, or about 700000, or about 710000, or about 720000, or about 730000, or about 740000, or about 750000, or about 760000, or about 770000, or about 780000, or about 790000, or about 800000, or about 810000, or about 820000, or about 830000, or about 840000, or about 850000, or about 860000, or about 870000, or about 880000, or about 890000, or about 900000, or about 910000, or about 920000, or about 930000, or about 940000, or about 950000, or about 960000, or about 970000, or about 980000, or about 990000, or about 1000000, or about 2000000, or about 2500000, or about 3000000, or about 3500000, or about 4000000, or about 4500000, or about 5000000, or about 5500000, or about 6000000, or about 6500000, or about 7000000, or about 7500000, or about 8000000, or about 8500000, or about 9000000, or about 9500000, or about 10000000 or more chemotherapeutic agents.

A PN-NP may, in some aspects, be functionalized with more than one chemotherapeutic agents that are different. The disclosure contemplates that a PN-NP comprises, in some embodiments, 2, 3, 4, 5, 6, 7, 8, 9, or 10 different chemotherapeutic agents. Any combination of chemotherapeutic agents may be attached to a PN-NP, and the various combinations can be determined by one of ordinary skill in the art.

In some embodiments, it is contemplated by the disclosure that the number of chemotherapeutic agents attached to a polynucleotide is in a ratio of one chemotherapeutic agent per nucleotide. Thus, in one aspect, a polynucleotide comprising 100 nucleotides can have 100 chemotherapeutic agents attached thereto. It will be understood that if more than one chemotherapeutic agent is attached to a polynucleotide, each additional chemotherapeutic agent(s) can be either the same or different than the first chemotherapeutic agent.

In some aspects, the chemotherapeutic agent is a platinum coordination complex, while in further aspects the platinum coordination complex is platinum(IV) (Pt(IV)). In some aspects, it is contemplated that a platinum(II) complex is attached to a polynucleotide through the equatorial ligands. For example and without limitation, CBDCA, the leaving group of carboplatin, can be functionalized at the cyclobutyl ring (malonate gamma position) with an ester moiety and attached to the NP functionalized in a compatible manner.

Accordingly, in some embodiments, the PN-NPs described herein are functionalized with thiolated polynucleotides containing a terminal dodecyl amine for conjugation. c,c,t-[Pt(NH₃)₂Cl₂(OH)(O₂CCH₂CH₂CO₂H)] (1) (Scheme 1) is a Pt(IV) compound capable of being tethered to an amine-functionalized DNA-Au NP surface via amide linkages [Di Pasqua et al., Mater. Lett. 63: 1876-1879 (2009)]. Treatment of 1 with 1-ethyl-3-[3-dimethylaminopropyl]carbodiimide (EDC) and N-hydroxysuccinimide (NHS) (see U.S. Pat. Nos. 6,806,289 and 7,651,979, incorporated by reference in their entirety) afforded its N-succinimidyl ester. This activated compound readily formed amide linkages with the amines on the PN-NP surface (Scheme 1), resulting in Pt(IV) loaded PN-NP conjugates (Pt-PN-NPs).

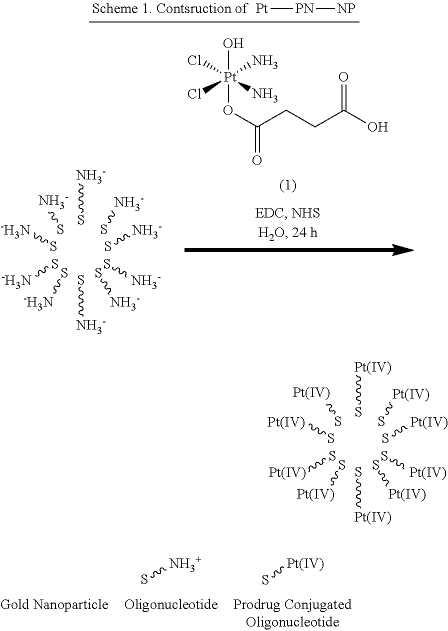

It will be understood that a chemotherapeutic agent can be attached to a polynucleotide in a multitude of ways, and these strategies are well known to those of skill in the art. In general, attachment is through an amide, ester or alkane. In aspects where the chemotherapeutic agent is platinum (Pt), it is contemplated that any attachment on the axial position of Pt, which will be released by reduction, may be used. Other attachment strategies for creating a PN-NP with an attached chemotherapeutic agent as described herein include adenylation, Acrydite™, cholesteryl-TEG, digoxigenin NHS Ester, I-Linker™, amino modifiers (including but not limited to amino modifier C6 and amino modifier C6 dT), biotinylation (including but not limited to biotin, biotin dT and biotin-TEG) and thiol modifications (including but not limited to thiol modifier C3 S—S, dithiol and thiol modifier Ch S—S). Additional methods of bioconjugate chemistry are detailed in Bioconjugate Techniques, 2nd Ed. by G. T. Hermanson. Academic Press, London, 1996, which is incorporated by reference herein in its entirety.

In various aspects, the chemotherapeutic agent includes those described herein below. In some embodiments, each polynucleotide attached to a nanoparticle comprises the same chemotherapeutic agent attached thereto. In other embodiments, separate polynucleotides on a nanoparticle each comprise a different chemotherapeutic agent attached thereto. Various combinations are contemplated by the disclosure, and are understood by one of skill in the art.

### Gene Regulatory Capacity of a Polynucleotide of the Disclosure

In various aspects, the polynucleotide that is attached to the nanoparticle is single-stranded. In some aspects, the polynucleotide that is attached to the nanoparticle is double-stranded. It will be appreciated that, in various aspects, both the polynucleotide and second polynucleotide, as described herein, may be either single or double-stranded. In various aspects wherein the polynucleotide or second polynucleotide that is attached to the nanoparticle is double-stranded, one strand of the double-stranded polynucleotide is a guide strand.

Guide strands (Scheme 2, dashed strands) are polynucleotide sequences designed to be complementary (antisense) to transcribed RNAs of any expressed (which, in some aspects, is upregulated) protein in, for example and without limitation, any human malignancy as determined by prior investigations. Sequences that are complementary to these guide strands (Scheme 2 solid strands) are synthesized and attached to thiolated O-ethylene glycol (OEG) (Scheme 2, bolded solid strands) and loaded onto the NP surface. Guide strands are then duplexed to thiolated OEG strands to produce the final product (Scheme 2).

Polynucleotides contemplated for attachment to a nanoparticle include those which modulate expression of a gene product expressed from a target polynucleotide. In some aspects, the polynucleotide that modulates expression of a gene product expressed from a target polynucleotide is not attached to a nanoparticle. The polynucleotides may, in various aspects, be comprised of DNA or RNA. Accordingly, antisense polynucleotides which hybridize to a target polynucleotide and inhibit translation, siRNA polynucleotides which hybridize to a target polynucleotide and initiate an RNAse activity (for example but not limited to RNAse H), triple helix forming polynucleotides which hybridize to double-stranded polynucleotides and inhibit transcription, and ribozymes which hybridize to a target polynucleotide and inhibit translation, are contemplated. Another polynucleotide contemplated for use in the compositions and according to the methods described herein is an aptamer.

In some embodiments, the polynucleotide that is attached to the nanoparticle is an antagomiR. An antagomiR represents a novel class of chemically engineered polynucleotides. AntagomiRs are used to silence endogenous microRNA (miRNA) [Krützfeldt et al., Nature 438 (7068): 685-9 (2005)]. AntagomiRs are, in some aspects, covalently modified with lipophoilic groups (for example and without limitation, cholesterol), or other agents specifically used to image the location of the antagomiR (for example and without limitation, a detectable marker as described herein). It is also contemplated that a composition of the disclosure comprises, in some aspects, an antagomiR that is not attached to a nanoparticle.

In various aspects, if a specific mRNA is targeted, a single nanoparticle-binding agent composition has the ability to hind to multiple copies of the same transcript. In one aspect, a nanoparticle is provided that is functionalized with identical polynucleotides, i.e., each polynucleotide has the same length and the same sequence. In other aspects, the nanoparticle is functionalized with two or more polynucleotides which are not identical, i.e., at least one of the attached polynucleotides differ from at least one other attached polynucleotide in that it has a different length and/or a different sequence. In aspects wherein different polynucleotides are attached to the nanoparticle, these different polynucleotides bind to the same single target polynucleotide but at different locations, or substrate sites, or bind to different target polynucleotides which encode different gene products. Accordingly, in various aspects, a single nanoparticle-binding agent composition target more than one gene product. Polynucleotides are thus target-specific polynucleotides, whether at one or more specific regions in the target polynucleotide, or over the entire length of the target polynucleotide as the need may be to effect a desired level of inhibition of gene expression.

### Target Polynucleotide Sequences and Hybridization

In some aspects, the disclosure provides methods of targeting a specific polynucleotide. It is contemplated that any polynucleotide that is attached to a nanoparticle or is otherwise in a composition as described herein may contribute to modulation of gene expression by associating with a target polynucleotide. Thus, the polynucleotide that contributes to modulation of gene expression through association with a target polynucleotide can be attached to the nanoparticle, wherein it may or may not include a chemotherapeutic agent, or it can be associated with the nanoparticle, or it can be delivered separately either as part of a targeting moiety or as a free polynucleotide.

Any type of polynucleotide may be targeted, and the methods may be used, e.g., for therapeutic modulation of gene expression (See, e.g., PCT/US2006/022325, the disclosure of which is incorporated herein by reference). Examples of polynucleotides that can be targeted by the methods of the invention include but are not limited to genes (e.g., a gene associated with a particular disease), viral RNA, mRNA, RNA, or single-stranded nucleic acids.

The target nucleic acid may be in cells as described herein.

The terms “start codon region” and “translation initiation codon region” refer to a portion of a mRNA or gene that encompasses contiguous nucleotides in either direction (i.e., 5′ or 3′) from a translation initiation codon. Similarly, the terms “stop codon region” and “translation termination codon region” refer to a portion of such a mRNA or gene that encompasses contiguous nucleotides in either direction (i.e., 5′ or 3′) from a translation termination codon. Consequently, the “start codon region” (or “translation initiation codon region”) and the “stop codon region” (or “translation termination codon region”) are all regions which may be targeted effectively with the polynucleotides on the functionalized nanoparticles.

Other target regions include the 5′ untranslated region (5′UTR), the portion of an mRNA in the 5′ direction from the translation initiation codon, including nucleotides between the 5′ cap site and the translation initiation codon of a mRNA (or corresponding nucleotides on the gene), and the 3′ untranslated region (3′UTR), the portion of a mRNA in the 3′ direction from the translation termination codon, including nucleotides between the translation termination codon and 3′ end of a mRNA (or corresponding nucleotides on the gene). The 5′ cap site of a mRNA comprises an N7-methylated guanosine residue joined to the 5′-most residue of the mRNA via a 5′-5′ triphosphate linkage. The 5′ cap region of a mRNA is considered to include the 5′ cap structure itself as well as the first 50 nucleotides adjacent to the cap site.

For prokaryotic target nucleic acid, in various aspects, the nucleic acid is RNA transcribed from genomic DNA. For eukaryotic target nucleic acid, the nucleic acid is an animal nucleic acid, a fungal nucleic acid, including yeast nucleic acid. As above, the target nucleic acid is a RNA transcribed from a genomic DNA sequence. In certain aspects, the target nucleic acid is a mitochondrial nucleic acid. For viral target nucleic acid, the nucleic acid is viral genomic RNA, or RNA transcribed from viral genomic DNA.

In some embodiments of the disclosure, a target polynucleotide sequence is a microRNA. MicroRNAs (miRNAs) are 20-22 nucleotide (nt) molecules generated from longer 70-nt RNAs that include an imperfectly complementary hairpin segment [Jackson et al., Sci STKE 367: rel (2007); Mendell, Cell Cycle 4: 1179-1184 (2005)]. The longer precursor molecules are cleaved by a group of proteins (Drosha and DCGR8) in the nucleus into smaller RNAs called pre-miRNA. Pre-miRNAs are then exported into the cytoplasm by exportin [Virmani et al., J Vasc Intery Radiol 19: 931-936 (2008)] proteins. The pre-miRNA in the cytoplasm is then cleaved into mature RNA by a complex of proteins called RNAi silencing complex or RISC. The resulting molecule has 19-bp double-stranded RNA and 2 nt 3′ overhangs on both strands. One of the two strands is then expelled from the complex and is degraded. The resulting single strand RNA-protein complex can then inhibit translation (either by repressing the actively translating ribosomes or by inhibiting initiation of translation) or enhance degradation of the mRNA it is attached to. There is, of course, a high degree of selectivity to this process, as the miRNA only binds to areas that are of high match to its sequence [Zamore et al., Science 309: 1519-1524 (2005)]. In one aspect, the target polynucleotide is microRNA-210.

Methods for inhibiting gene product expression provided include those wherein expression of the target gene product is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% compared to gene product expression in the absence of an polynucleotide-functionalized nanoparticle. In other words, methods provided embrace those which results in essentially any degree of inhibition of expression of a target gene product.

The degree of inhibition is determined in vivo from a body fluid sample or from a biopsy sample or by imaging techniques well known in the art. Alternatively, the degree of inhibition is determined in a cell culture assay, generally as a predictable measure of a degree of inhibition that can be expected in vivo resulting from use of a specific type of nanoparticle and a specific polynucleotide.

### Modified Polynucleotides

Modified polynucleotides are contemplated for functionalizing nanoparticles wherein both one or more sugar and/or one or more internucleotide linkage of the nucleotide units in the polynucleotide is replaced with “non-naturally occurring” groups. In one aspect, this embodiment contemplates a peptide nucleic acid (PNA). In PNA compounds, the sugar-backbone of a polynucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

Other linkages between nucleotides and unnatural nucleotides contemplated for the disclosed polynucleotides include those described in U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920; U.S. Patent Publication No. 20040219565; International Patent Publication Nos. WO 98/39352 and WO 99/14226; Mesmaeker et. al., Current Opinion in Structural Biology 5:343-355 (1995) and Susan M. Freier and Karl-Heinz Altmann, Nucleic Acids Research, 25:4429-4443 (1997), the disclosures of which are incorporated herein by reference.

Specific examples of polynucleotides include those containing modified backbones or non-natural internucleoside linkages. Polynucleotides having modified backbones include those that retain a phosphorus atom in the backbone and those that do not have a phosphorus atom in the backbone. Modified polynucleotides that do not have a phosphorus atom in their internucleoside backbone are considered to be within the meaning of “polynucleotide.”

Modified polynucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are polynucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated.

Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified polynucleotide backbones that do not include a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. In still other embodiments, polynucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In various forms, the linkage between two successive monomers in the oligo consists of 2 to 4, desirably 3, groups/atoms selected from —CH₂—, —O—, —S—, —NRH—, >C═O, >C═NRH, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHRH)—, where RH is selected from hydrogen and C1-4-alkyl, and R″ is selected from C1-6-alkyl and phenyl. Illustrative examples of such linkages are —CH₂—CH₂—CH₂—, —CH₂—CO—CH₂—, —CH₂—CHOH—CH₂—, —O—CH2—O—, —O—CH2—CH2—, —O—CH2—CH=(including R5 when used as a linkage to a succeeding monomer), —CH₂—CH₂—O—, —NRH—CH₂—CH₂—, —CH₂—CH₂—NRH—, —CH₂—NRH—CH₂—, —O—CH₂—CH₂—NRH—, —NRH—CO—O—, —NRH—CO—NRH—, —NRH—CS—NRH—, —NRH—C(═NRH)—NRH—, —NRH—CO—CH₂—NRH—O—CO—O—, —O—CO—CH₂—O—, —O—CH₂—CO—O—, —CH₂—CO—NRH—, —O—CO—NRH—, —NRH—CO—CH₂—, —O—CH₂—CO—NRH—, —O—CH₂—CH₂—NRH—, —CH═N—O—, —CH₂—NRH—O—, —CH₂—O—N=(including R5 when used as a linkage to a succeeding monomer), —CH₂—O—NRH—, —CO—NRH—CH₂—, —CH₂—NRH—O—, —CH₂—NRH—CO—, —O—NRH—CH₂—, —O—NRH—, —O—CH₂—S—, —S—CH₂—O—, —CH₂—CH₂—S—, —O—CH₂—CH₂—S—, —S—CH₂—CH=(including R5 when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂CH₂—, —O—S(O)₂—NRH—, —NRH—S(O)₂—CH₂—; —O—S(O)₂—CH₂—, —O—P(O)₂—O—, —O—P(O,S)—O—, —O—P(S)₂—O—, —S—P(O)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(O CH₂CH₃)—O—, —O—PO(O CH₂CH₂S—R)—O—, —O—PO(BH₃)—O—, —O—PO(NHRN)—O—, —O—P(O)₂—NRH H—, —NRH—P(O)₂—O—, —O—P(O,NRH)—O—, —CH₂—P(O)₂—O—, —O—P(O)₂—CH₂—, and —O—Si(R″)₂—O—; among which —CH₂—CO—NRH—, —CH₂—NRH—O—, —S—CH₂—O—, —O—P(O)₂—O—O—P(—O,S)—O—, —O—P(S)₂—O—, —NRH P(O)₂—O—, —O—P(O,NRH)—O—, —O—PO(R″)—O—, —O—PO(CH₃)—O—, and —O—PO(NHRN)—O—, where RH is selected form hydrogen and C1-4-alkyl, and R″ is selected from C₁₋₆-alkyl and phenyl, are contemplated. Further illustrative examples are given in Mesmaeker et. al., 1995, Current Opinion in Structural Biology, 5: 343-355 and Susan M. Freier and Karl-Heinz Altmann, 1997, Nucleic Acids Research, vol 25: pp 4429-4443.

Still other modified forms of polynucleotides are described in detail in U.S. Patent Application No. 20040219565, the disclosure of which is incorporated by reference herein in its entirety.

Modified polynucleotides may also contain one or more substituted sugar moieties. In certain aspects, polynucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH2)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other polynucleotides comprise one of the following at the 2′ position: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a polynucleotide, or a group for improving the pharmacodynamic properties of a polynucleotide, and other substituents having similar properties. In one aspect, a modification includes 2′-methoxyethoxy (2′-O—CH₂CH₂OCH₃, also known as 2′-O-(2-methoxyethyl) or 2′-MOE) (Martin et al., 1995, Helv. Chim. Acta, 78: 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CHₛ)₂.

Still other modifications include 2′-methoxy (2′—O—CH₃), 2′-aminopropoxy (2′-OCH₂CH₂CH₂NH₂), 2′-allyl (2′-CH₂—CH═CH₂), 2′ O-allyl (2′O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the polynucleotide, for example, at the 3′ position of the sugar on the 3′ terminal nucleotide or in 2′-5′ linked polynucleotides and the 5′ position of 5′ terminal nucleotide. Polynucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In one aspect, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects a methylene (—CH₂—)n group bridging the 2′ oxygen atom and the 4′ carbon atom wherein n is 1 or 2. LNAs and preparation thereof are described in WO 98/39352 and WO 99/14226, the disclosures of which are incorporated herein by reference.

### Methods of Attaching Polynucleotides

Polynucleotides contemplated for use in the methods include those bound to the nanoparticle through any means. Regardless of the means by which the polynucleotide is attached to the nanoparticle, attachment in various aspects is effected through a 5′ linkage, a 3′ linkage, some type of internal linkage, or any combination of these attachments.

In one aspect, the nanoparticles, the polynucleotides or both are functionalized in order to attach the polynucleotides to the nanoparticles. Methods to functionalize nanoparticles and polynucleotides are known in the art. For instance, polynucleotides functionalized with alkanethiols at their 3′-termini or 5′-termini readily attach to gold nanoparticles. See Whitesides, Proceedings of the Robert A. Welch Foundation 39th Conference On Chemical Research Nanophase Chemistry, Houston, Tex., pages 109-121 (1995). See also, Mucic et al. [Chem. Commun. 555-557 (1996)] which describes a method of attaching 3′ thiol DNA to flat gold surfaces. The alkanethiol method can also be used to attach polynucleotides to other metal, semiconductor and magnetic colloids and to the other types of nanoparticles described herein. Other functional groups for attaching polynucleotides to solid surfaces include phosphorothioate groups (see, for example, U.S. Pat. No. 5,472,881 for the binding of polynucleotide-phosphorothioates to gold surfaces), substituted alkylsiloxanes [(see, for example, Burwell, Chemical Technology, 4, 370-377 (1974) and Matteucci and Caruthers, J. Am. Chem. Soc., 103, 3185-3191 (1981) for binding of polynucleotides to silica and glass surfaces, and Grabar et al., [Anal. Chem., 67, 735-743] for binding of aminoalkylsiloxanes and for similar binding of mercaptoaklylsiloxanes]. Polynucleotides with a 5′ thionucleoside or a 3′ thionucleoside may also be used for attaching polynucleotides to solid surfaces. The following references describe other methods which may be employed to attach polynucleotides to nanoparticles: Nuzzo et al., J. Am. Chem. Soc., 109, 2358 (1987) (disulfides on gold); Allara and Nuzzo, Langmuir, 1, 45 (1985) (carboxylic acids on aluminum); Allara and Tompkins, J. Colloid Interface Sci., 49, 410-421 (1974) (carboxylic acids on copper); Iler, The Chemistry Of Silica, Chapter 6, (Wiley 1979) (carboxylic acids on silica); Timmons and Zisman, J. Phys. Chem., 69, 984-990 (1965) (carboxylic acids on platinum); Soriaga and Hubbard, J. Am. Chem. Soc., 104, 3937 (1982) (aromatic ring compounds on platinum); Hubbard, Acc. Chem. Res., 13, 177 (1980) (sulfolanes, sulfoxides and other functionalized solvents on platinum); Hickman et al., J. Am. Chem. Soc., 111, 7271 (1989) (isonitriles on platinum); Maoz and Sagiv, Langmuir, 3, 1045 (1987) (silanes on silica); Maoz and Sagiv, Langmuir, 3, 1034 (1987) (silanes on silica); Wasserman et al., Langmuir, 5, 1074 (1989) (silanes on silica); Eltekova and Eltekov, Langmuir, 3, 951 (1987) (aromatic carboxylic acids, aldehydes, alcohols and methoxy groups on titanium dioxide and silica); Lec et al., J. Phys. Chem., 92, 2597 (1988) (rigid phosphates on metals).

U.S. patent application Ser. Nos. 09/760,500 and 09/820,279 and international application nos. PCT/US01/01190 and PCT/US01/10071 describe polynucleotides functionalized with a cyclic disulfide. The cyclic disulfides in certain aspects have 5 or 6 atoms in their rings, including the two sulfur atoms. Suitable cyclic disulfides are available commercially or are synthesized by known procedures. Functionalization with the reduced forms of the cyclic disulfides is also contemplated. Functionalization with triple cyclic disulfide anchoring groups are described in PCT/US2008/63441, incorporated herein by reference in its entirety.

In certain aspects wherein cyclic disulfide functionalization is utilized, polynucleotides are attached to a nanoparticle through one or more linkers. In one embodiment, the linker comprises a hydrocarbon moiety attached to a cyclic disulfide. Suitable hydrocarbons are available commercially, and are attached to the cyclic disulfides. The hydrocarbon moiety is, in one aspect, a steroid residue. Polynucleotide-nanoparticle compositions prepared using linkers comprising a steroid residue attached to a cyclic disulfide are more stable compared to compositions prepared using alkanethiols or acyclic disulfides as the linker, and in certain instances, the polynucleotide-nanoparticle compositions have been found to be 300 times more stable. In certain embodiments the two sulfur atoms of the cyclic disulfide are close enough together so that both of the sulfur atoms attach simultaneously to the nanoparticle. In other aspects, the two sulfur atoms are adjacent each other. In aspects where utilized, the hydrocarbon moiety is large enough to present a hydrophobic surface screening the surfaces of the nanoparticle.

In other aspects, a method for attaching polynucleotides onto a surface is based on an aging process described in U.S. application Ser. No. 09/344,667, filed Jun. 25, 1999; Ser. No. 09/603,830, filed Jun. 26, 2000; Ser. No. 09/760,500, filed Jan. 12, 2001; Ser. No. 09/820,279, filed Mar. 28, 2001; Ser. No. 09/927,777, filed Aug. 10, 2001; and in International application nos. PCT/US97/12783, filed Jul. 21, 1997; PCT/US00/17507, filed Jun. 26, 2000; PCT/US01/01190, filed Jan. 12, 2001; PCT/US01/10071, filed Mar. 28, 2001, the disclosures which are incorporated by reference in their entirety. The aging process provides nanoparticle-polynucleotide compositions with enhanced stability and selectivity. The process comprises providing polynucleotides, in one aspect, having covalently bound thereto a moiety comprising a functional group which can bind to the nanoparticles. The moieties and functional groups are those that allow for binding (i.e., by chemisorption or covalent bonding) of the polynucleotides to nanoparticles. For example, polynucleotides having an alkanethiol, an alkanedisulfide or a cyclic disulfide covalently bound to their 5′ or 3′ ends bind the polynucleotides to a variety of nanoparticles, including gold nanoparticles.

Compositions produced by use of the “aging” step have been found to be considerably more stable than those produced without the “aging” step. Increased density of the polynucleotides on the surfaces of the nanoparticles is achieved by the “aging” step. The surface density achieved by the “aging” step will depend on the size and type of nanoparticles and on the length, sequence and concentration of the polynucleotides. A surface density adequate to make the nanoparticles stable and the conditions necessary to obtain it for a desired combination of nanoparticles and polynucleotides can be determined empirically. Generally, a surface density of at least 2 picomoles/cm² will be adequate to provide stable nanoparticle-polynucleotide compositions. Regardless, various polynucleotide densities are contemplated as disclosed herein.

An “aging” step is incorporated into production of functionalized nanoparticles following an initial binding or polynucleotides to a nanoparticle. In brief, the polynucleotides are contacted with the nanoparticles in water for a time sufficient to allow at least some of the polynucleotides to bind to the nanoparticles by means of the functional groups. Such times can be determined empirically. In one aspect, a time of about 12-24 hours is contemplated. Other suitable conditions for binding of the polynucleotides can also be determined empirically. For example, a concentration of about 10-20 nM nanoparticles and incubation at room temperature is contemplated.

Next, at least one salt is added to the water to form a salt solution. The salt is any water-soluble salt, including, for example and without limitation, sodium chloride, magnesium chloride, potassium chloride, ammonium chloride, sodium acetate, ammonium acetate, a combination of two or more of these salts, or one of these salts in phosphate buffer. The salt is added as a concentrated solution, or in the alternative as a solid. In various embodiments, the salt is added all at one time or the salt is added gradually over time. By “gradually over time” is meant that the salt is added in at least two portions at intervals spaced apart by a period of time. Suitable time intervals can be determined empirically.

The ionic strength of the salt solution must be sufficient to overcome at least partially the electrostatic repulsion of the polynucleotides from each other and, either the electrostatic attraction of the negatively-charged polynucleotides for positively-charged nanoparticles, or the electrostatic repulsion of the negatively-charged polynucleotides from negatively-charged nanoparticles. Gradually reducing the electrostatic attraction and repulsion by adding the salt gradually over time gives the highest surface density of polynucleotides on the nanoparticles. Suitable ionic strengths can be determined empirically for each salt or combination of salts. In one aspect, a final concentration of sodium chloride of from about 0.01 M to about 1.0 M in phosphate buffer is utilized, with the concentration of sodium chloride being increased gradually over time. In another aspect, a final concentration of sodium chloride of from about 0.01 M to about 0.5 M, or about 0.1 M to about 0.3 M is utilized, with the concentration of sodium chloride being increased gradually over time.

After adding the salt, the polynucleotides and nanoparticles are incubated in the salt solution for a period of time to allow additional polynucleotides to bind to the nanoparticles to produce the stable nanoparticle-polynucleotide compositions. An increased surface density of the polynucleotides on the nanoparticles stabilizes the compositions, as has been described herein. The time of this incubation can be determined empirically. By way of example, in one aspect a total incubation time of about 24-48, wherein the salt concentration is increased gradually over this total time, is contemplated. This second period of incubation in the salt solution is referred to herein as the “aging” step. Other suitable conditions for this “aging” step can also be determined empirically. By way of example, an aging step is carried out with incubation at room temperature and pH 7.0.

The compositions produced by use of the “aging” are in general more stable than those produced without the “aging” step. As noted above, this increased stability is due to the increased density of the polynucleotides on the surfaces of the nanoparticles which is achieved by the “aging” step. The surface density achieved by the “aging” step will depend on the size and type of nanoparticles and on the length, sequence and concentration of the polynucleotides.

As used herein, “stable” means that, for a period of at least six months after the compositions are made, a majority of the polynucleotides remain attached to the nanoparticles and the polynucleotides are able to hybridize with nucleic acid and polynucleotide targets under standard conditions encountered in methods of detecting nucleic acid and methods of nanofabrication.

In some aspects, it is contemplated that RNA is functionalized on a nanoparticle. Methods of attaching RNA to a nanoparticle are described in WO/2010/060110, the disclosure of which is incorporated herein by reference in its entirety.

### Surface Density

Nanoparticles as provided herein have a packing density of the polynucleotides on the surface of the nanoparticle that is, in various aspects, sufficient to result in cooperative behavior between nanoparticles and between polynucleotide strands on a single nanoparticle.

In another aspect, the cooperative behavior between the nanoparticles increases the resistance of the polynucleotide to nuclease degradation. In yet another aspect, the uptake of nanoparticles by a cell is influenced by the density of polynucleotides associated with the nanoparticle. As described in PCT/US2008/65366, incorporated herein by reference in its entirety, a higher density of polynucleotides on the surface of a nanoparticle is associated with an increased uptake of nanoparticles by a cell.

A surface density adequate to make the nanoparticles stable and the conditions necessary to obtain it for a desired combination of nanoparticles and polynucleotides can be determined empirically. Generally, a surface density of at least 2 pmoles/cm² will be adequate to provide stable nanoparticle-polynucleotide compositions. In some aspects, the surface density is at least 15 pmoles/cm². Methods are also provided wherein the polynucleotide is bound to the nanoparticle at a surface density of at least 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm² at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more.

Density of polynucleotides on the surface of a nanoparticle has been shown to modulate specific polypeptide interactions with the polynucleotide on the surface and/or with the nanoparticle itself. Under various conditions, some polypeptides may be prohibited from interacting with polynucleotides associated with a nanoparticle based on steric hindrance caused by the density of polynucleotides. In aspects where interaction of polynucleotides with polypeptides that are otherwise precluded by steric hindrance is desirable, the density of polynucleotides on the nanoparticle surface is decreased to allow the polypeptide to interact with the polynucleotide.

Polynucleotide surface density has also been shown to modulate stability of the polynucleotide associated with the nanoparticle. In one embodiment, an RNA polynucleotide associated with a nanoparticle is provided wherein the RNA polynucleotide has a half-life that is at least substantially the same as the half-life of an identical RNA polynucleotide that is not associated with a nanoparticle. In other embodiments, the RNA polynucleotide associated with the nanoparticle has a half-life that is about 5% greater, about 10% greater, about 20% greater, about 30% greater, about 40% greater, about 50% greater, about 60% greater, about 70% greater, about 80% greater, about 90% greater, about 2-fold greater, about 3-fold greater, about 4-fold greater, about 5-fold greater, about 6-fold greater, about 7-fold greater, about 8-fold greater, about 9-fold greater, about 10-fold greater, about 20-fold greater, about 30-fold greater, about 40-fold greater, about 50-fold greater, about 60-fold greater, about 70-fold greater, about 80-fold greater, about 90-fold greater, about 100-fold greater, about 200-fold greater, about 300-fold greater, about 400-fold greater, about 500-fold greater, about 600-fold greater, about 700-fold greater, about 800-fold greater, about 900-fold greater, about 1000-fold greater, about 5000-fold greater, about 10,000-fold greater, about 50,000-fold greater, about 100,000-fold greater, about 200,000-fold greater, about 300,000-fold greater, about 400,000-fold greater, about 500,000-fold greater, about 600,000-fold greater, about 700,000-fold greater, about 800,000-fold greater, about 900,000-fold greater, about 1,000,000-fold greater or more than the half-life of an identical RNA polynucleotide that is not associated with a nanoparticle.

### Polynucleotide Features

The present disclosure provides, in various embodiments, PN-NP compositions that are useful for gene regulation. In some aspects, the PN-NP is functionalized with DNA. In some embodiments, the DNA is double-stranded, and in further embodiments the DNA is single-stranded. In further aspects, the PN-NP is functionalized with RNA, and in still further aspects the PN-NP is functionalized with double-stranded RNA agents known as small interfering RNA (siRNA). The term “RNA” includes duplexes of two separate strands, as well as single-stranded structures. Single-stranded RNA also includes RNA with secondary structure. In one aspect, RNA having a hairpin loop in contemplated.

Polynucleotides that are contemplated for use in gene regulation and functionalized to a nanoparticle have complementarity to (i.e., are able to hybridize with) a portion of a target RNA (generally messenger RNA (mRNA)).

“Hybridization” means an interaction between two or three strands of nucleic acids by hydrogen bonds in accordance with the rules of Watson-Crick complementarity, Hoogstein binding, or other sequence-specific binding known in the art. Hybridization can be performed under different stringency conditions known in the art.

Generally, such complementarity is 100%, but can be less if desired, such as about 20%, about 25%, about 30%, about 35%, about 40%, about 45%, about 50%, about 55%, about 60%, about 70%, about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. For example, 19 bases out of 21 bases may be base-paired. Thus, it will be understood that a polynucleotide used in the methods need not be 100% complementary to a desired target nucleic acid to be specifically hybridizable. Moreover, polynucleotides may hybridize to each other over one or more segments such that intervening or adjacent segments are not involved in the hybridization event (e.g., a loop structure or hairpin structure). Percent complementarity between any given polynucleotide can be determined routinely using BLAST programs (Basic Local Alignment Search Tools) and PowerBLAST programs known in the art (Altschul et al., 1990, J. Mol. Biol., 215: 403-410; Zhang and Madden, 1997, Genome Res 7: 649-656).

In some aspects, where selection between various allelic variants is desired, 100% complementarity to the target gene is required in order to effectively discern the target sequence from the other allelic sequence. When selecting between allelic targets, choice of length is also an important factor because it is the other factor involved in the percent complementary and the ability to differentiate between allelic differences.

### Detectable Marker

Methods are provided wherein presence of a polynucleotide is detected by an observable change. In one aspect, presence of the polynucleotide gives rise to a color change which is observed with a device capable of detecting a specific marker as disclosed herein. For example and without limitation, a fluorescence microscope can detect the presence of a fluorophore that is conjugated to a polynucleotide, which has been functionalized on a nanoparticle.

It will be understood that a marker contemplated will include any of the fluorophores described herein as well as other detectable markers known in the art. For example, markers also include, but are not limited to, redox active probes, other nanoparticles, metabolic groups and quantum dots, as well as any marker which can be detected using spectroscopic means, i.e., those markers detectable using microscopy and cytometry. In various aspects, isotopes are contemplated as a general method of identifying the location of embolized material as described below. In further aspects, imaging contrast agents (for example and without limitation, gadolinium and/or fluorine) are contemplated as a general method of identifying the location of a composition of the disclosure.

Suitable fluorescent molecules are also well known in the art and include without limitation 1,8-ANS (1-Anilinonaphthalene-8-sulfonic acid), 1-Anilinonaphthalene-8-sulfonic acid (1,8-ANS), 5-(and -6)-Carboxy-T, T-dichlorofluorescein pH 9.0, 5-FAM pH 9.0, 5-ROX (5-Carboxy-X-rhodamine, triethylammonium salt), 5-ROX pH 7.0, 5-TAMRA, 5-TAMRA pH 7.0, 5-TAMRA-MeOH, 6 JOE, 6,8-Difluoro-7-hydroxy-4-methylcoumarin pH 9.0, 6-Carboxyrhodamine 6G pH 7.0, 6-Carboxyrhodamine 6G, hydrochloride, 6-HEX, SE pH 9.0, 6-TET, SE pH 9.0, 7-Amino-4-methylcoumarin pH 7.0, 7-Hydroxy-4-methylcoumarin, 7-Hydroxy-4-methylcoumarin pH 9.0, Alexa 350, Alexa 405, Alexa 430, Alexa 488, Alexa 532, Alexa 546, Alexa 555, Alexa 568, Alexa 594, Alexa 647, Alexa 660, Alexa 680, Alexa 700, Alexa Fluor 430 antibody conjugate pH 7.2, Alexa Fluor 488 antibody conjugate pH 8.0, Alexa Fluor 488 hydrazide-water, Alexa Fluor 532 antibody conjugate pH 7.2, Alexa Fluor 555 antibody conjugate pH 7.2, Alexa Fluor 568 antibody conjugate pH 7.2, Alexa Fluor 610 R-phycoerythrin streptavidin pH 7.2, Alexa Fluor 647 antibody conjugate pH 7.2, Alexa Fluor 647 R-phycoerythrin streptavidin pH 7.2, Alexa Fluor 660 antibody conjugate pH 7.2, Alexa Fluor 680 antibody conjugate pH 7.2, Alexa Fluor 700 antibody conjugate pH 7.2, Allophycocyanin pH 7.5, AMCA conjugate, Amino Coumarin, APC (allophycocyanin), Atto 647, BCECF pH 5.5, BCECF pH 9.0, BFP (Blue Fluorescent Protein), BO-PRO-1-DNA, BO-PRO-3-DNA, BOBO-1-DNA, BOBO-3-DNA, BODIPY 650/665-X, MeOH, BODIPY FL conjugate, BODIPY FL, MeOH, Bodipy R6G SE, BODIPY R6G, MeOH, BODIPY TMR-X antibody conjugate pH 7.2, Bodipy TMR-X conjugate, BODIPY TMR-X, MeOH, BODIPY TMR-X, SE, BODIPY TR-X phallacidin pH 7.0, BODIPY TR-X, MeOH, BODIPY TR-X, SE, BOPRO-1, BOPRO-3, Calcein, Calcein pH 9.0, Calcium Crimson, Calcium Crimson Ca2+, Calcium Green, Calcium Green-1 Ca2+, Calcium Orange, Calcium Orange Ca2+, Carboxynaphthofluorescein pH 10.0, Cascade Blue, Cascade Blue BSA pH 7.0, Cascade Yellow, Cascade Yellow antibody conjugate pH 8.0, CFDA, CFP(Cyan Fluorescent Protein), CI-NERF pH 2.5, CI-NERF pH 6.0, Citrine, Coumarin, Cy 2, Cy 3, Cy 3.5, Cy 5, Cy 5.5, CyQUANT GR-DNA, Dansyl Cadaverine, Dansyl Cadaverine, MeOH, DAPI, DAPI-DNA, Dapoxyl (2-aminoethyl) sulfonamide, DDAO pH 9.0, Di-8 ANEPPS, Di-8-ANEPPS-lipid, Dil, DiO, DM-NERF pH 4.0, DM-NERF pH 7.0, DsRed, DTAF, dTomato, eCFP (Enhanced Cyan Fluorescent Protein), eGFP (Enhanced Green Fluorescent Protein), Eosin, Eosin antibody conjugate pH 8.0, Erythrosin-5-isothiocyanate pH 9.0, Ethidium Bromide, Ethidium homodimer, Ethidium homodimer-1-DNA, eYFP (Enhanced Yellow Fluorescent Protein), FDA, FITC, FITC antibody conjugate pH 8.0, FlAsH, Fluo-3, Fluo-3 Ca2+, Fluo-4, Fluor-Ruby, Fluorescein, Fluorescein 0.1 M NaOH, Fluorescein antibody conjugate pH 8.0, Fluorescein dextran pH 8.0, Fluorescein pH 9.0, Fluoro-Emerald, FM 1-43, FM 1-43 lipid, FM 4-64, FM 4-64, 2% CHAPS, Fura Red Ca2+, Fura Red, high Ca, Fura Red, low Ca, Fura-2 Ca2+, Fura-2, high Ca, Fura-2, no Ca, GFP(S65T), HcRed, Hoechst 33258, Hoechst 33258-DNA, Hoechst 33342, Indo-1 Ca2+, Indo-1, Ca free, Indo-1, Ca saturated, JC-1, JC-1 pH 8.2, Lissamine rhodamine, LOLO-1-DNA, Lucifer Yellow, CH, LysoSensor Blue, LysoSensor Blue pH 5.0, LysoSensor Green, LysoSensor Green pH 5.0, LysoSensor Yellow pH 3.0, LysoSensor Yellow pH 9.0, LysoTracker Blue, LysoTracker Green, LysoTracker Red, Magnesium Green, Magnesium Green Mg2+, Magnesium Orange, Marina Blue, mBanana, mCherry, mHoneydew, MitoTracker Green, MitoTracker Green FM, MeOH, MitoTracker Orange, MitoTracker Orange, MeOH, MitoTracker Red, MitoTracker Red, MeOH, mOrange, mPlum, mRFP, mStrawberry, mTangerine, NBD-X, NBD-X, MeOH, NeuroTrace 500/525, green fluorescent Niss1 stain-RNA, Nile Blue, EtOH, Nile Red, Nile Red-lipid, Niss1, Oregon Green 488, Oregon Green 488 antibody conjugate pH 8.0, Oregon Green 514, Oregon Green 514 antibody conjugate pH 8.0, Pacific Blue, Pacific Blue antibody conjugate pH 8.0, Phycoerythrin, PicoGreen dsDNA quantitation reagent, PO-PRO-1, PO-PRO-1-DNA, PO-PRO-3, PO-PRO-3-DNA, POPO-1, POPO-1-DNA, POPO-3, Propidium Iodide, Propidium Iodide-DNA, R-Phycoerythrin pH 7.5, ReAsH, Resorufin, Resorufin pH 9.0, Rhod-2, Rhod-2 Ca2+, Rhodamine, Rhodamine 110, Rhodamine 110 pH 7.0, Rhodamine 123, MeOH, Rhodamine Green, Rhodamine phalloidin pH 7.0, Rhodamine Red-X antibody conjugate pH 8.0, Rhodaminen Green pH 7.0, Rhodol Green antibody conjugate pH 8.0, Sapphire, SBFI-Na+, Sodium Green Na+, Sulforhodamine 101, EtOH, SYBR Green I, SYPRO Ruby, SYTO 13-DNA, SYTO 45-DNA, SYTOX Blue-DNA, Tetramethylrhodamine antibody conjugate pH 8.0, Tetramethylrhodamine dextran pH 7.0, Texas Red-X antibody conjugate pH 7.2, TO-PRO-1-DNA, TO-PRO-3-DNA, TOTO-1-DNA, TOTO-3-DNA, TRITC, X-Rhod-1 Ca2+, YO-PRO-1-DNA, YO-PRO-3-DNA, YOYO-1-DNA, and YOYO-3-DNA.

In yet another embodiment, two types of fluorescent-labeled polynucleotides attached to two different particles can be used. This may be useful, for example and without limitation, to track two different cell populations. Suitable particles include polymeric particles (such as, without limitation, polystyrene particles, polyvinyl particles, acrylate and methacrylate particles), glass particles, latex particles, Sepharose beads and others like particles well known in the art. Methods of attaching polynucleotides to such particles are well known and routinely practiced in the art. See Chrisey et al., 1996, Nucleic Acids Research, 24: 3031-3039 (glass) and Charreyre et al., 1997 Langmuir, 13: 3103-3110, Fahy et al., 1993, Nucleic Acids Research, 21: 1819-1826, Elaissari et al., 1998, J. Colloid Interface Sci., 202: 251-260, Kolarova et al., 1996, Biotechniques, 20: 196-198 and Wolf et al., 1987, Nucleic Acids Research, 15: 2911-2926 (polymer/latex).

Other labels besides fluorescent molecules can be used, such as chemiluminescent molecules, which will give a detectable signal or a change in detectable signal upon hybridization.

Methods of labeling polynucleotides with fluorescent molecules and measuring fluorescence are well known in the art.

### Chemotherapeutic Agents

Compositions and methods of the present disclosure relate to a polynucleotide functionalized nanoparticle, wherein a chemotherapeutic agent is attached to the polynucleotide. In one specific embodiment, the chemotherapeutic agent is a platinum coordination complex, and in further aspects the platinum coordination complex is platinum(IV) (Pt(IV)) or is platinum(II) (Pt(II)).

According to the disclosure, chemotherapeutic agents useful in the compositions and methods includes those that are activated or become therapeutically effective upon entry into a cell. The activation, in various aspects, results from reduction of a chemotherapeutic agent, cleavage of a prodrug to result in its active form, activation resulting from binding of the chemotherapeutic agent to a binding partner, enzymatic cleavage of an appropriately designed linker functionality like an ester, hydrolysis in an intracellular compartment such as an endosome, or any other change that occurs as a result of the chemotherapeutic having entered the cell.

In some embodiments, activation of the chemotherapeutic agent upon entry into a cell results in a relative increase in activity of about 1% compared to the activity of the chemotherapeutic agent prior to entry into a cell. In various aspects, activation of the chemotherapeutic agent upon entry into a cell results in a relative increase in activity of about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 2-fold, about 3-fold, about 4-fold, about 5-fold, about 6-fold, about 7-fold, about 8-fold, about 9-fold, about 10-fold, about 15-fold, about 20-fold, about 25-fold, about 30-fold, about 40-fold, about 50-fold, about 60-fold, about 70-fold, about 80-fold, about 90-fold, about 100-fold or more compared to the activity of the chemotherapeutic agent prior to entry into a cell.

Additional chemotherapeutic agents contemplated for use include, without limitation, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2″-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics such as actimomycin D, daunomycin (rubidomycin), doxorubicin, mitoxantrone, idarubicin, bleomycins, plicamycin (mithramycin), mitomycinC, and actinomycin; enzymes such as L-asparaginase; biological response modifiers such as interferon-alpha, IL-2, G-CSF and GM-CSF; miscellaneous agents including platinum coordination complexes such as cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p″-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

### Activation of a Chemotherapeutic Agent

According to the disclosure, it is contemplated that a chemotherapeutic agent that is attached to a PN-NP as described herein is activated upon entry into a cell. In some aspects, the activated chemotherapeutic agent confers an increase in cytotoxicity relative to a chemotherapeutic agent that is not attached to a polynucleotide, wherein the polynucleotide is functionalized on a nanoparticle, and wherein the increase in cytotoxicity is measured using an in vitro cell culture assay. The in vitro cell culture assay is, for example and without limitation, a (3-(4,5-dimethylthiazol-2-yl)-2,5-diphenyltetrazolium bromide) (MTT) assay. Accordingly, the increase in cytotoxicity described above is coupled with the reduced toxicity of the chemotherapeutic agent which is attached to a polynucleotide that is functionalized on a nanoparticle prior to its entry into a cell.

The increase in cytotoxicity, in one aspect, is about 2-fold relative to a chemotherapeutic agent that is not attached to a polynucleotide, wherein the polynucleotide is functionalized on a nanoparticle, and wherein the increase in cytotoxicity is measured using an in vitro cell culture assay. In further aspects, the increase in cytotoxicity is about 3-fold, about 4-fold, about 5-fold, about 6-fold, about 7-fold, about 8-fold, about 9-fold, about 10-fold, about 100-fold, about 1000-fold, about 10,000-fold, about 100,000-fold, about 1,000,000-fold or higher relative to a chemotherapeutic agent that is not attached to a polynucleotide, wherein the polynucleotide is functionalized on a nanoparticle, and wherein the increase in cytotoxicity is measured using an in vitro cell culture assay.

### Therapeutic Agents

In some embodiments, a composition of the present disclosure further comprises a therapeutic agent. In some aspects, the therapeutic agent is associated with the nanoparticle. In other aspects, the therapeutic agent is co-administered with the PN-NP, but is separate from the PN-NP composition. The therapeutic agent is, in various aspects, administered before, concurrent with, or after the administration of the PN-NP composition. One of ordinary skill in the art will understand that multiple therapeutic agents in multiple combinations can be administered at any time before, concurrent with or after administration of the PN-NP composition. In addition, repeated administration of a therapeutic agent is also contemplated.

In an embodiment of the invention, the therapeutic agent is selected from the group consisting of a protein, peptide, a small molecule, a radioactive material, and a polynucleotide.

Protein therapeutic agents include, without limitation peptides, enzymes, structural proteins, receptors and other cellular or circulating proteins as well as fragments and derivatives thereof, the aberrant expression of which gives rise to one or more disorders. Therapeutic agents also include, as one specific embodiment, chemotherapeutic agents. Still other therapeutic agents include polynucleotides, including without limitation, protein coding polynucleotides, polynucleotides encoding regulatory polynucleotides, and/or polynucleotides which are regulatory in themselves. Therapeutic agents also include, in various embodiments, a radioactive material.

In various aspects, protein therapeutic agents include cytokines or hematopoietic factors including without limitation IL-1 alpha, IL-1 beta, IL-2, IL-3, IL-4, IL-5, IL-6, IL-11, colony stimulating factor-1 (CSF-1), M-CSF, SCF, GM-CSF, granulocyte colony stimulating factor (G-CSF), EPO, interferon-alpha (IFN-alpha), consensus interferon, IFN-beta, IFN-gamma, IL-7, IL-8, IL-9, IL-10, IL-12, IL-13, IL-14, IL-15, IL-16, IL-17, IL-18, thrombopoietin (TPO), angiopoietins, for example Ang-1, Ang-2, Ang-4, Ang-Y, the human angiopoietin-like polypeptide, vascular endothelial growth factor (VEGF), angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor, cytokine-induced neutrophil chemotactic factor 1, cytokine-induced neutrophil, chemotactic factor 2a, cytokine-induced neutrophil chemotactic factor 2β, β endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor al, glial cell line-derived neutrophic factor receptor α2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor receptor, TNF, including TNF0, TNF1, TNF2, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type urokinase-type plasminogen activator receptor, vascular endothelial growth factor, and chimeric proteins and biologically or immunologically active fragments thereof.

The term “small molecule,” as used herein, refers to a chemical compound, for instance a peptidometic or polynucleotide that may optionally be derivatized, or any other low molecular weight organic compound, either natural or synthetic. Such small molecules may be a therapeutically deliverable substance or may be further derivatized to facilitate delivery.

By “low molecular weight” is meant compounds having a molecular weight of less than 1000 Daltons, typically between 300 and 700 Daltons. Low molecular weight compounds, in various aspects, are about 100, about 150, about 200, about 250, about 300, about 350, about 400, about 450, about 500, about 550, about 600, about 650, about 700, about 750, about 800, about 850, about 900, about 1000 or more Daltons.

Polynucleotide therapeutic agents include, in one aspect and without limitation, those which encode therapeutic proteins described herein and otherwise known in the art, as well as polynucleotides which have intrinsic regulatory functions. Polynucleotides that have regulatory functions have been described herein above and include without limitation RNAi, antisense, ribozymes, and triplex-forming polynucleotides, each of which have the ability to regulate gene expression. Methods for carrying out these regulatory functions have previously been described in the art (Dykxhoom D M, Novina C D and Sharp P A, Nature Review, 4: 457-467, 2003; Mittal V, Nature Reviews, 5: 355-365, 2004).

### Target Site Identification and Composition Delivery

Methods provided include those wherein a composition of the disclosure is delivered to a target cell. As described herein above, in some embodiments a composition is preferentially delivered to a target cell via a targeting moiety. It is also contemplated that a composition is delivered locally to a target site, with or without a targeting moiety. Once the target site has been identified, a composition of the disclosure is delivered, in one aspect, intraarterially. In other aspects, a composition of the disclosure is delivered intravenously, orally or intraperitoneally. In further aspects, a composition of the disclosure is delivered in combination with an embolic agent. In various aspects of the compositions and methods of the disclosure, the embolic agent is selected from the group consisting of a lipid emulsion (for example and without limitation, ethiodized oil or lipiodol), gelatin sponge, tris acetyl gelatin microspheres, embolization coils, ethanol, small molecule drugs, biodegradable microspheres, non-biodegradable microspheres or polymers, and self-assemblying embolic material. In further aspects, delivery further comprises administration of an additional embolic agent. Target site identification is performed, in some aspects, by interventional radiology.

A target cell is located at the target site. In some aspects, the target cell is a cancer cell, and in further aspects the cancer is selected from the group consisting of liver, pancreatic, stomach, colorectal, prostate, testicular, renal cell, breast, bladder, ureteral, brain, lung, connective tissue, hematological, cardiovascular, lymphatic, skin, bone, eye, nasopharyngeal, laryngeal, esophagus, oral membrane, tongue, thyroid, parotid, mediastinum, ovary, uterus, adnexal, endometrial, cervical, small bowel, appendix, carcinoid, gall bladder, pituitary, cancer arising from metastatic spread, and cancer arising from endodermal, mesodermal or ectodermally-derived tissues.

In one specific aspect, the disclosure provides compositions that comprise a platinum (Pt) coordination complex that is less active but is activated when attached to PN-NPs. These Pt-PN-NPs are internalized by cells and reduced to release cisplatin, which enters the nucleus and forms 1,2-d(GpG) intrastrand cross-links with DNA, resulting in cytotoxicity.

In various embodiments, a second administration of a composition described herein is delivered. In some aspects, the second delivery is administered 24 hours after delivering the composition. In various aspects, the second delivery is administered about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, or about 60 minutes after delivering the composition. In further aspects, the second delivery is administered about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, or about 24 hours after delivering the composition. In still further aspects, the second delivery is administered about 1.5 days, about 2 days, about 3 days, about 4 days, about 5 days, about 6 days, about 7 days, about 8 days, about 9 days, about 10 days, about 11 days, about 12 days, about 13 days, about 2 weeks, about 2.5 weeks, about 3 weeks, about 3.5 weeks, about 4 weeks, about 4.5 weeks, about 5 weeks, about 5.5 weeks, about 6 weeks, about 6.5 weeks, about 7 weeks, about 7.5 weeks, about 8 weeks, about 8.5 weeks, about 9 weeks, about 9.5 weeks, about 10 weeks or more after delivering the composition.

It will be appreciated that, in various aspects, a therapeutic agent as described herein is attached to the nanoparticle.

### EXAMPLES

### Example 1

### Construction of PN-NP and Chemotherapeutic Agent

The complexes cis-[Pt(NH₃)₂Cl₂] [Dhara, Indian J. Chem. 8: 193-194 (1970)] and c,c,t-[Pt(NH₃)₂Cl₂(OH)₂] [Hall et al., J. Biol. Inorg. Chem. 8: 726-732 (2003)] were synthesized as previously described. Distilled water was purified by passage through a Millipore Milli-Q Biocel water purification system (18.2 MΩ) containing a 0.22 μM filter. The detection of the cisplatin 1,2-d(GpG) intrastrand adduct was carried out by immunofluorescence with the use of a monoclonal adduct-specific antibody R-C18 (provided by Dr. Jürgen Thomale, University of Duisburg-Essen). FITC labeled secondary antibody rabbit anti-(rat Ig) was obtained from Invitrogen. Specific adhesion slides for immunofluoresecence were purchased from Squarix Biotechnology, Marl, Germany. Atomic absorption spectroscopic measurements were taken on a Perkin Elmer AAnalyst 600 spectrometer. Fluorescence imaging studies were performed with a DeltaVision deconvolution microscope.

### Synthesis of c,c,t-[Pt(NH₃)₂Cl₂(OH)(O₂CCH₂CH₂CO₂H)](1)

To a solution of c,c,t-[Pt(NH₃)₂Cl₂(OH)₂] (0.2 g, 0.6 mmol) in DMSO (16 ml) was added succinic anhydride (0.06 g, 0.6 mmol) and the reaction mixture was stirred at room temperature for 12 hours. The solution was lyophilized and 10 mL acetone was added to precipitate a light yellow solid, which was washed several times with acetone, diethyl ether, and then dried. c,c,t-[Pt(NH₃)₂Cl₂(OH)(O₂CCH₂CH₂CO₂H)] (I) was isolated in 54% (0.14 g) yield. ESI-MS (M-H) Calcd.=434.98. Found=434.0. ¹H NMR (DMSO-d6) 6.52 (br, 6H), 2.97-1.97 (m, 4H).

### Synthesis of PN-NP

13±1 nm Au NPs were synthesized by the Frens method [Frens, Nature-Phys. Sci. 241: 20-22 (1973)], resulting in approximately 10 nM solutions. The polynucleotides used to functionalize the Au NP were 5′ dodecyl amine-TAG CTG CAC GCT GCC GTC-((CH₂CH₂O)₆PO₃)₂-propylthiol 3′ (SEQ ID NO: 1) and 5′ Cy5-TAG CTG CAC GCT GCC GTC-((CH₂CH₂O)₆PO₃)₂-propylthiol 3′ (SEQ ID NO: 2). They were prepared with standard phosphoramidite reagents purchased from Glen Research, purified by reverse phase HPLC, and characterized by MALDI (amine terminated polynucleotide: Calcd: 6648. Found: 6649; Cy5 terminated polynucleotide: Calcd: 6917. Found: 6920). The polynucleotide Au NP conjugates were synthesized as described previously [Hurst et al., Anal. Chem. 78: 8313-8318 (2006)]. Briefly, polynucleotides were mixed with the as-synthesized Au NPs at a concentration of 2 μM. Tween 20, phosphate buffer, pH=7.4, and NaCl were added sequentially to the solution to final concentrations of 0.01% (v/v), 10 mM, and 1.0 M, respectively. The mixture was sonicated for 1 min and then incubated overnight at room temperature.

The particles were purified by repeated centrifugation and resuspension in a solution of 0.15 M NaCl and 10 mM phosphate buffer. The mixed monolayer particles were synthesized similarly, except that the amine and Cy5 terminated polynucleotides were used at a concentration of 1 μM each.

### Synthesis of Pt-PN-NP

The synthesis of Pt-PN-NP was carried out by using standard amide coupling reactions. In a typical reaction, a 1.0 mM aqueous solution of NHS (20 μL) was added to an equal volume of an aqueous 1.0 mM solution of EDC and the resulting solution was allowed to stand at room temperature for 10 minutes. To this solution was added 0.8 molar equivalents of compound I in ddH2O (40 μL). After 10 minutes, a solution of DNA-Au NP was added; the mole ratio of amine-to-Pt was 0.5. The solution was stirred for 24 hours at room temperature. The resulting particles were purified from excess 1 using 100 kDa molecular weight cutoff ultracentrifugation filtration membranes. The concentration Pt-DNA-Au NP was subsequently determined by platinum atomic absorption spectroscopy (AAS).

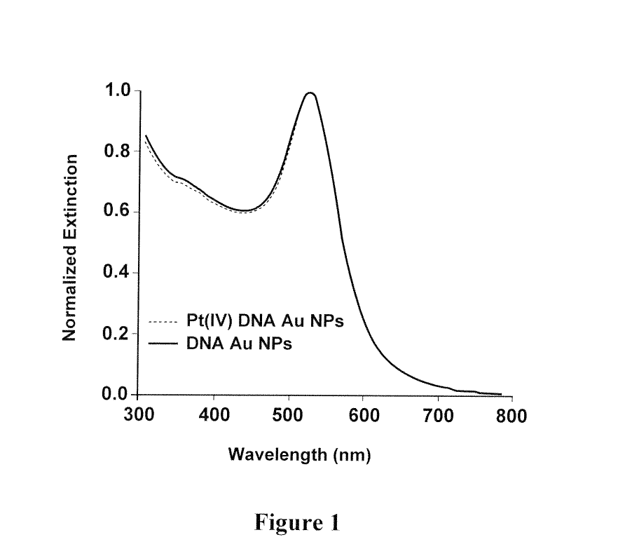

The PN-NPs used in this study were thus functionalized with thiolated 28-mer polynucleotides containing a terminal dodecyl amine for conjugation. c,c,t-[Pt(NH₃)₂Cl₂(OH)(O₂CCH₂CH₂—CO₂H)] (1) (Scheme 1) was used, a Pt(IV) compound capable of being tethered to an amine-functionalized PN-NP surface via amide linkages. Treatment of 1 with 1-ethyl-3[3-dimethylaminopropyl]carbodiimide (EDC) and N-hydroxysuccinimide (NHS) afforded its N-succinimidyl ester. This activated compound readily formed amide linkages with the amines on the PN-NP surface (Scheme 1), resulting in Pt(IV) loaded DNA-Au NP conjugates (Pt-PN-NPs). The resulting particles were purified from excess 1 using 100 kDa molecular weight cutoff ultracentrifugation filtration membranes. The Pt-PN-NP conjugates were characterized by platinum AAS, which showed that 98% of the PN-NP amines were conjugated to platinum. Importantly, UV-vis spectroscopy of the Pt-PN-NPs surface plasmon band confirmed that there is no aggregation of the particles after prodrug conjugation (FIG. 1).

The conjugate was designed to release a cytotoxic dose of cisplatin upon intracellular reduction. An ideal Pt(IV) complex should be sufficiently stable to travel through the blood stream until it reaches a tumor cell without decomposition. Once inside the cell, however, it should have an appropriate reduction potential such that it will be reduced and release its cytotoxic payload. Electrochemical measurements were made at 25° C. on a EG&G PAR Model 263 Potentiostat/Galvanostat with electrochemical analysis software 270 and a three electrode set-up comprising a glassy carbon working electrode, platinum wire auxiliary electrode and a Ag/AgCl reference electrode. The electrochemical data were uncorrected for junction potentials. KCl was used as a supporting electrolyte.

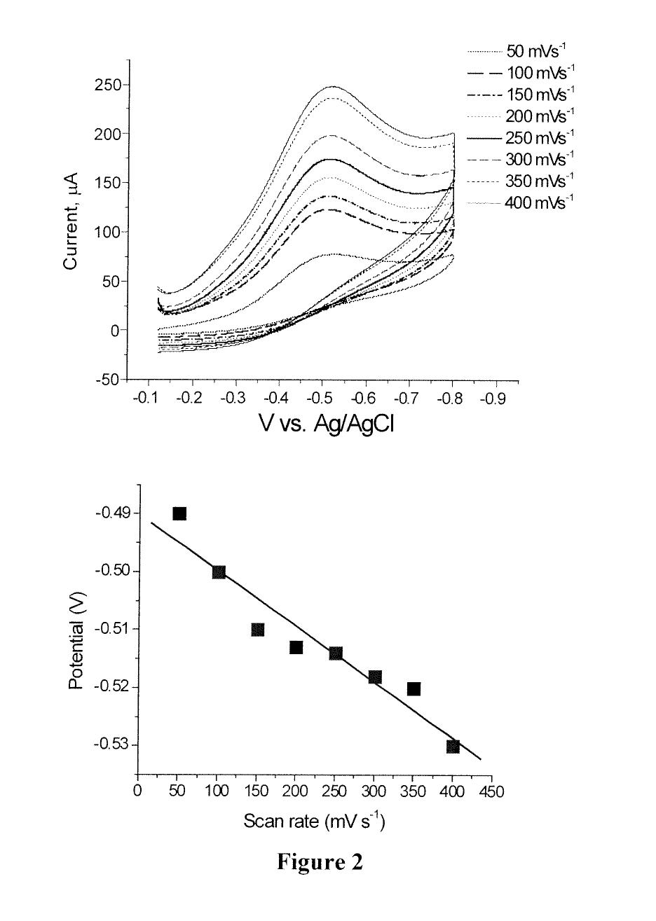

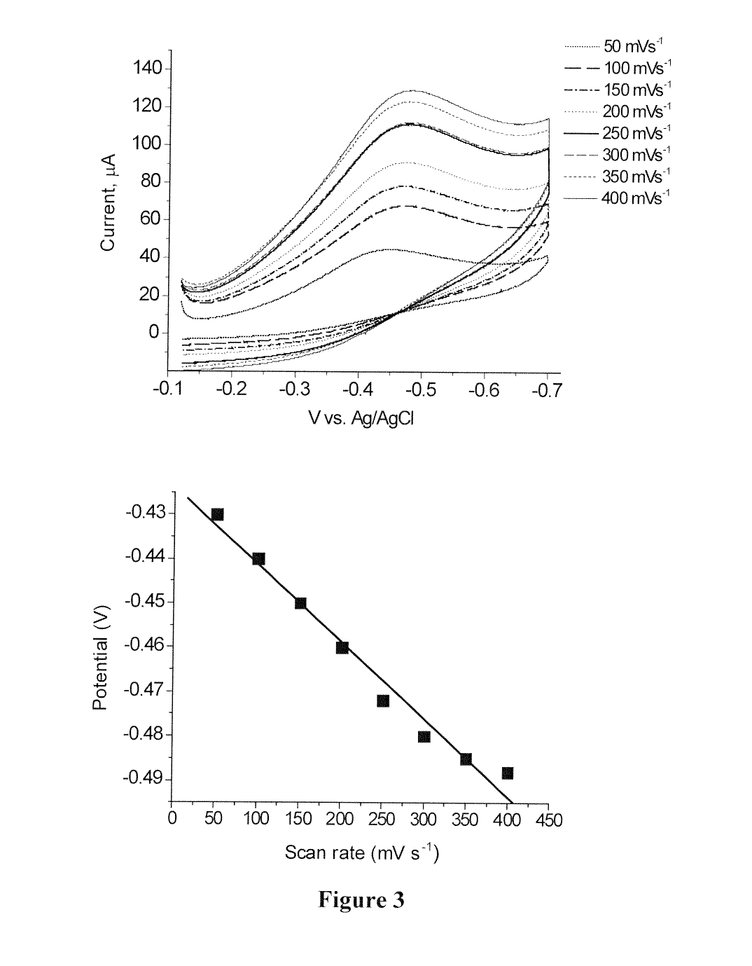

Electrochemical studies of 1 revealed an irreversible reduction maximum, corresponding to loss of the axial ligands (FIGS. 2 and 3). At pH 7.4, the reduction potential of 1 when extrapolated to a 0.0 mV s-1 scan rate is −0.49 V. At pH 6.0, a value similar to that reported for endosomes [Arunachalam et al., Proc. Natl. Acad. Sci. U.S.A. 97: 745-750 (2000)], there is a positive shift of its reduction potential to −0.42 V, indicating that the acidic environment in cancer cells will facilitate reduction of the complex. The conjugation of 1 to the Au NP surface via an amide bond is not expected to significantly alter the reduction potential of the Pt(IV) center.

### Example 2

### Uptake of Pt-PN-NPs

Human cervical cancer HeLa, human osteosarcoma U2OS, human prostate PC3 cell lines were procured from the ATCC. A549 lung carcinoma cells were obtained from Prof. David E. Root, Whitehead Institute for Biomedical Research. HeLa, U2OS, and A549 Cells were grown at 37° C. in 5% CO₂ in DMEM medium supplemented with 10% fetal bovine serum and 1% penicillin/streptomycin. PC3 cells were grown at 37° C. in 5% CO₂ in RPMI supplemented with 10% fetal bovine serum and 1% penicillin/streptomycin. Cells were passed every 3 to 4 days and restarted from frozen stocks upon reaching pass number 20.

The ability of Pt-DNA-Au NPs to enter cells was investigated by fluorescence microscopy using particles functionalized with a mixed monolayer of platinated polynucleotide strands and 5′ dye (Cy5) labeled strands. HeLa cells were grown in EMEM with 10% heat inactivated fetal bovine serum and maintained at 37° C. in 5% CO₂. Cells were seeded in 12 well chamber plates and grown for 24 hours prior to transfection with 10 pM of Pt-DNA-Au NP conjugate which were labeled with Cy5 DNA mixed on the surface. Live cells were imaged 6 hours and 12 hours post-treatment using a 60× objective on a DeltaVision deconvolution microscope (Applied Precision). Hoechst 33342 was used to provide nuclear staining. Oregon Green 488 taxol bis acetate was used to stain microtubules.

The conjugates were incubated with cells for varying periods of time. After 6 hours, the conjugates had localized in vesicles, and after 12 hours, particles were observed in the cytosol. Oregon Green 488® taxol bis acetate, which stains microtubules, indicated co-localization of these conjugates with the microtubules in HeLa cells.

In order to investigate the efficacy of Pt-DNA-Au NPs, their ability to kill cancer cells of various origin was measured. An MTT assay was applied to measure cytotoxicity induced by Pt-DNA-Au NPs and the activity was compared to those of free cisplatin and the parent Pt(IV) compound I. All the solutions for the MTT assay were freshly prepared in sterile PBS before use and quantitated by atomic absorption spectroscopy. Cells were seeded on a 96 well plate in 100 μL media and incubated for 24 hours. The cells were then treated with Pt-DNA-Au NP, 1, or cisplatin, separately at varying concentrations and incubated for 72 hours at 37° C. Medium was removed, cells were washed with PBS and then incubated with cell culture medium containing 20% 3-(4,5-dimethylthiazol-2-yl)-2,5-diphenyltetrazolium bromide (MTT). After 3 hours incubation at 37° C. in 5% CO₂, 100 μL lysis buffer (20 grams SDS dissolved in 50 mL ddH₂O and supplemented with 50 mL N,N-Dimethylformamide, pH 4.7) per well was added. Cells were further incubated overnight and the absorbance was measured at 620 nm using a BioTek Synergy HT multi-detection microplate plate reader. Each condition was repeated in triplicate in three independent experiments for each cell line.

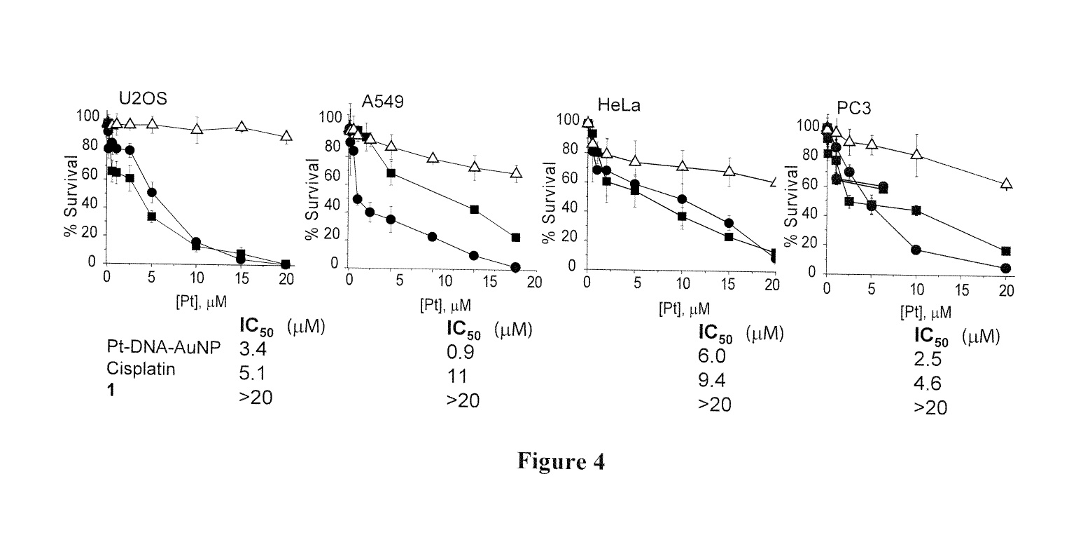

Cytotoxicity profiles of Pt-DNA-Au NPs in human lung carcinoma A549, human prostate cancer PC3, cervical cancer HeLa, and human osteosarcoma U2OS cells are shown in FIG. 4. After a 72 hour treatment with different concentrations of Pt-DNA-Au NPs, significant cytotoxicity was observed in all four cells lines. In A549 cells, cisplatin has an IC₅₀ of 11 μM, whereas that of the Pt-DNA-Au NP is 0.9 μM, indicating the superior killing ability of this conjugate compared to cisplatin for this cell type. The Pt-DNA-Au NP construct has an IC₅₀ value of 3.4 μM, comparable to that of cisplatin (IC₅₀, 5.1 μM), in U2OS cells. Similarly, in HeLa and PC3 cells, Pt-DNA-Au NP (IC₅₀ values 6.0 and 2.5 μM, respectively) reflect activity greater than that of cisplatin (IC₅₀ values 9.4 and 4.6 μM, respectively). The parent prodrug 1 did not show any significant killing under the same conditions. The enhanced activity of the Pt(IV) compound when tethered to a DNA-Au NP was an important finding of this study.

### Example 3

### Intrastrand 1,2-d(GpG) Formation

Detection of the platinum 1,2-d(GpG) adducts was carried out by following a procedure recently reported by us using an antibody specific to this adduct [Dhar et al., J. Am. Chem. Soc. 130: 11467-11476 (2008)]. Briefly, HeLa cells were seeded in a six well plate using DMEM medium and incubated overnight at 37° C. Pt-DNA-Au NP was added to a final concentration of 1 μM Pt and incubated at 37° C. After 12 hours, cells were trypsinized, washed with PBS, resuspended in HAES-sterile-PBS at a density of 1×10⁶ per mL and placed onto a pre-coated slide (ImmunoSelect, Squarix) and air dried. Cell fixing was carried out at −20° C. in methanol for 45 minutes. Nuclear DNA was denatured by alkali (70 mM NaOH, 140 mM NaCl, 40% methanol v/v) treatment for 5 minutes at 0° C., and cellular proteins were removed by proteolytic procedure involving two steps. The cells were first digested with pepsin at 37° C. for 10 minutes and then with proteinase K at 37° C. for 5 minutes. After blocking with milk (1% in PBS; 30 minutes; 25° C.) slides were incubated with anti-(Pt-DNA) monoclonal antibodies (Mabs) (R-C18 0.1 mg/mL in milk) [Liedert et al., Nucleic Acids Res. 34: e47 (2006)] for overnight at 4° C. After washing with PBS, immunostaining was performed by incubation with FITC-labeled goat anti-(rat Ig) antibody at 37° C. for 1 hour. The nuclei of the cells were stained by using Hoechst (H33258) (250 μg/L) and mounted using the mounting solution for imaging.

Since the anticancer activity of cisplatin derives from the formation of intrastrand 1,2-d(GpG) crosslinks on nuclear DNA, whether the platinum released following reduction of Pt-DNA-Au NP leads to this signature event was investigated by using a monoclonal antibody R-C18 specific for the adduct [Liedert et al., Nucleic Acids Res. 34: e47/1-e47/12 (2006)]. After a 48 hour incubation of Pt-DNA-Au NPs (1 μM) with HeLa cells, the formation of 1,2-d(GpG) intrastrand cross-links was observed by antibody-derived green fluorescence in the cell nuclei, confirming formation of Pt(II) 1,2-d(GpG) intrastrand crosslinks.

Thus, methods of attaching and delivering platinum compounds using DNA-Au NPs have been shown. A Pt(IV) complex, which is otherwise inactive, was made active against several cancer cell lines when attached to DNA-Au NPs. These conjugates are internalized by cells and reduced to release cisplatin, which enters the nucleus and forms 1,2-d(GpG) intrastrand cross-links with DNA. Pt-DNA-Au NPs are more effective than cisplatin in killing cancer cells of several kinds.

1. A composition comprising a nanoparticle functionalized with a polynucleotide (PN-NP) and a platinum coordination complex, wherein the platinum coordination complex is attached to the polynucleotide, and wherein the platinum coordination complex is activated upon cell uptake.

2. (canceled)

3. (canceled)

4. The composition of claim 1 wherein the nanoparticle is selected from the group consisting of a gold nanoparticle, a silver nanoparticle, a platinum nanoparticle, an aluminum nanoparticle, a palladium nanoparticle, a copper nanoparticle, a cobalt nanoparticle, an indium nanoparticle, an iron oxide nanoparticle and a nickel nanoparticle.

5. (canceled)

6. The composition of claim 1 wherein the platinum coordination complex is platinum(IV) (Pt(IV)) or platinum(II) (Pt(II)).

7. The composition of claim 1 wherein the activation results in an increase in cytotoxicity.

8. The composition of claim 7 wherein the increase in cytotoxicity is about 2-fold relative to a platinum coordination complex that is not attached to a polynucleotide, wherein the polynucleotide is functionalized on a nanoparticle, and wherein the increase in cytotoxicity is measured using an in vitro cell culture assay.

9. (canceled)

10. The composition of claim 1 wherein more than one platinum coordination complex is attached to the polynucleotide.

11. The composition of claim 1 wherein the polynucleotide is DNA, RNA, or a modified polynucleotide.

12. The composition of claim 1, wherein the polynucleotide comprises about 5 nucleotides to about 100 nucleotides.

13. (canceled)

14. (canceled)

15. The composition of claim 1 wherein the polynucleotide is double-stranded or single-stranded.

16. The composition of claim 1 further comprising a second polynucleotide.

17. The composition of claim 16 wherein the second polynucleotide is attached to the nanoparticle.

18. The composition of claim 16 wherein the second polynucleotide further comprises a detectable marker.

19. The composition of claim 18 wherein the detectable marker is selected from the group consisting of a fluorophore, an isotope, a contrast agent, a redox active probe, a nanoparticle, a polypeptide, a peptide, a small molecule, a metal, a metabolic group and a quantum dot.

20. The composition of claim 16 wherein the second polynucleotide is sufficiently complementary to a target polynucleotide to hybridize to the target polynucleotide.

21. The composition of claim 20 wherein the target polynucleotide is DNA or RNA.

22. The composition of claim 20 wherein the target polynucleotide is in a target cell.

23. The composition of claim 22 wherein the target cell is a cancer cell.

24. The composition of claim 23 wherein the cancer is selected from the group consisting of liver, pancreatic, stomach, colorectal, prostate, testicular, renal cell, breast, bladder, ureteral, brain, lung, connective tissue, hematological, cardiovascular, lymphatic, skin, bone, eye, nasopharyngeal, laryngeal, esophagus, oral membrane, tongue, thyroid, parotid, mediastinum, ovary, uterus, adnexal, endometrial, cervical, small bowel, appendix, carcinoid, gall bladder, pituitary, cancer arising from metastatic spread, and cancer arising from endodermal, mesodermal or ectodermally-derived tissues.

25. The composition of claim 16 wherein the polynucleotide and the second polynucleotide are each sufficiently complementary to hybridize to a different target polynucleotide in the target cell.

26. A method for delivering a platinum coordination complex to cytoplasm of a cell comprising administering the composition of claim 1 to the target cell under conditions and in an amount effective to deliver the platinum coordination complex to the cytoplasm of the cell.
